SEQUENCE LISTING (26 sequences)

SEQ ID NO: 1 - DNA, 121 bp, Lycopersicon esculentum
  polymorphism (61)..(61): susceptible = C; tolerant = T
gtcggaccaagaaaccatatttggtaacgggttcgagttgctgcctgaaccttttagcccyttgcaatatttgtgaagtgatattcctttgtgttattaataatttttcgttttgagtttt

SEQ ID NO: 2 - DNA, 121 bp, Lycopersicon esculentum
  polymorphism (61)..(61): susceptible = T; tolerant = C
ttcagagagcaacactcctgcaagaccaactcggagtaattcagtaactcgaccttccatytctagctctcagtatagtacttactcaaataaatcaggctctattctaaacacaagctct

SEQ ID NO: 3 - DNA, 101 bp, Lycopersicon esculentum
  polymorphism (51)..(51): susceptible = A; tolerant = G
ttccgaaatgaggacgatccatcagcttcttcagctgagagcccctggtcracataccagaattctgtttttctaaaactgtccaaaatctcctgtaaaga

SEQ ID NO: 4 - DNA, 71 bp, Lycopersicon esculentum
  polymorphism (36)..(36): susceptible = T; tolerant = C
gatttgaatgccttgccacagccagaggatgacgaygagatttttggacaacaattagaagatgaaccaca

SEQ ID NO: 5 - DNA, 121 bp, Lycopersicon esculentum
  polymorphism (61)..(61): susceptible = A; tolerant = G
agagaatgatatcactgccttagtttctcaattaaaagttgtgcaaaaacaaaacacacarctagatgaagaaaacagagcattcgcctcaaagcttcagacaaaagaagttgagaacaac

SEQ ID NO: 6 - DNA, 71 bp, Lycopersicon esculentum
  polymorphism (36)..(36): susceptible = T; tolerant = G
tacaataccttctggcatccctttccgcaaaacgakagatctttagtatcaaaaccgagagcactgtcacc

SEQ ID NO: 7 - DNA, 101 bp, Lycopersicon esculentum
  polymorphism (51)..(51): susceptible = A; tolerant = T
atggcaatagtgaactgcagatacaactgaaattgcagaacacccttaaawatagaatcaatagaaagttgcaacaatatttgaatgatgaagcaacaaag

SEQ ID NO: 8 - DNA, 101 bp, Lycopersicon esculentum
  polymorphism (51)..(51): susceptible = T; tolerant = C
agccattgtgattgtgtctgttgtacattaccaaaattctctagagaaagygatacacatgccagccctatcgatataaagcaacgcaaggtggattctgc

SEQ ID NO: 9 - DNA, 101 bp, Lycopersicon esculentum
  polymorphism (51)..(51): susceptible = C; tolerant = T
aacaccaggtagagagcacagcgaaacaatggcctcaggaagatctacttygcgaagtgcagcaagccactccatacctccaccaggctttgatttcagtg

SEQ ID NO: 10 - DNA, 101 bp, Lycopersicon esculentum
  polymorphism (51)..(51): susceptible = A; tolerant = G
tcaactgcaactttaacagctgattcaacttcttcttctttcgaaacatcrcattgaatgtaacgacctccaatagattcagctaaacttgtacctacttc

SEQ ID NO: 11 - DNA, 101 bp, Lycopersicon esculentum
  polymorphism (51)..(51): susceptible = C; tolerant = A
gaggagctatcaacttcatagtcagattcagaaaatgattcagatgaggamgtggctgattcttcttgttttcttttcttccttctgctcgaactctctcc

SEQ ID NO: 12 - DNA, 101 bp, Lycopersicon esculentum
  polymorphism (51)..(51): susceptible = G; tolerant = A
cagaaataatagaaaatcagaaagaaaaatcagctttctaaatggaaaagrcgatggcactatgtttgaagttttaagcaacttttctgaagtcccaaaag

SEQ ID NO: 13 - DNA, 160 bp, Lycopersicon esculentum
  polymorphism (59)..(59): susceptible = A; tolerant = G
ctcctattgaacatcctgaaaacttgtgtctacatcatgagaagatgcaggccaattcrctcagtacatggaatgcacgagcatgttaggggaattctaacgcaaagcataagcttgatacttgaataaaagatgaaacatacttacttcttctcaaact

SEQ ID NO: 14 - DNA, 101 bp, Lycopersicon esculentum
  polymorphism (51)..(51): susceptible = A; tolerant = G
ctcttggtgacaaaccactggctcaatttcttcgcgaagctaaagctatcrctgatgagcttgtcacggcaggcacacgtgtctcctgatgaattcaatgc

SEQ ID NO: 15 - DNA, 101 bp, Lycopersicon esculentum
  polymorphism (51)..(51): susceptible = T; tolerant = G
cattactgttgagatatctcatcggcaacccctggagcttgcccacccgcktgtcctccaggatctgatttcagaaaggatgaatagtaactgtgtttcag

SEQ ID NO: 16 - DNA, 121 bp, Lycopersicon esculentum
  polymorphism (61)..(61): susceptible = G; tolerant = A
caagaacccaacgacttcttcttctttgcttattgaaaaacttggttttgaaatgaaaggratcgagaaattggatactcagtggttctctactactaaaccttctcctgattttaagaaa

SEQ ID NO: 17 - DNA, 101 bp, Lycopersicon esculentum
  polymorphism (51)..(51): susceptible = T; tolerant = C
tgatcgacaattcttgttgttgttgaaactctgcaagtgagagagggatgyatatagagaaaggatattggtaaaggacaattctagaagggtctagggaa

SEQ ID NO: 18 - DNA, 101 bp, Lycopersicon esculentum
  polymorphism (51)..(51): susceptible = C; tolerant = T
gcaccagttatagtaatgtcctgcttctttcctgtacccttatcagtagcygtgacagaaagaataccgttggtgtcaatgtcgaacttcacttcaatctg

SEQ ID NO: 19 - protein, 754 aa, Lycopersicon esculentum
MATAQSNSPRVFCIGTADTKFDELRFLSEHVRSSLNSFSNKSSFKVGVTVVDVSTSWKETNSCADFDFVPSKDVLSCHTLGEETMGTFADIRGLAIAIMSKALETFLSIANDEQNLAGVIGLGGSGGTSLLSSAFRSLPIGIPKVIISTVASGQTESYIGTSDLVLFPSVVDICGINNVSKVVLSNAGAAFAGMVIGRLESSKEHSITNGKFTVGVTMFGVTTPCVNAVKERLVKEGYETLVFHATGVGGRAMEDLVRGGFIQGVLDITTTEVADYVVGGVMACDSSRFDAILEKKIPLVLSVGALDMVNFGPKTTIPPEFQQRKIHEHNEQVSLMRTTVGENKKFAAFIAEKLNKASSSVCVCLPEKGVSALDAPGKDFYDPEATSCLTRELQMLLENNERCQVKVLPYHINDAEFANALVDSFLEISPKSRHVECQPAESKSIQDIQNDNAVLEKYPSCNGKNFSRLNDFPNAKPETLQKRTVILQKLKDQISKGKPIIGAGAGTGISAKFEEAGGVDLIVLYNSGRFRMAGRGSLAGLLPFADANAIVLEMANEVLPVVKEVAVLAGVCATDPFRRMDNFLKQLESVGFCGVQNFPTVGLFDGNFRQNLEETGMGYGLEVEMIAAAHRMGLLTTPYAFCPDEAVAMAEAGADIIVAHMGLTTSGSIGAKTAVSLEESVTCVQAIADATHRIYPDAIVLCHGGPISSPEEAAYVLKRTTGVHGFYGASSMERLPVEQAITATVQQYKSISME

SEQ ID NO: 20 - DNA, 2718 bp, Lycopersicon esculentum
gacttttttatttatttaatattaaatattaatattgctccatcggaataaaaataaaaataaaaatggaacctctccacttgacggttgttgccgcctctctgtccaaccgttgccggtgatctttcatcgtcgagattccagaagctaacaactttctaatcttctcaccattctcacactgagttacactctatcgtccattttgaaatctcgattgtaacacgaattgtatcattttgtttgaggaaaatggcaactgcacagagtaattctcctcgagttttctgtatcggaacagctgatacaaaattcgacgagcttcgtttcctctccgagcatgtgagatccagtcttaacagcttctccaataaatcctcattcaaggtaggagtgacagttgttgatgtctcaaccagctggaaggagacaaatagttgtgctgattttgattttgtaccgagtaaggatgtgctgtcatgccatacactaggggaagaaactatgggcacgtttgcagatattagaggcctagctattgcaatcatgagcaaagctcttgaaactttcctaagtatagctaatgatgaacagaatcttgctggagtaattggccttgggggtagtggaggaacatctctattgtcatctgccttccgatctcttccaattgggatcccaaaagttataatatctacagttgccagtggtcaaactgaatcttatattggaacatcagacttggtattgtttccttcagttgtagatatttgtgggattaacaatgtcagtaaggttgttctatctaatgcgggtgcagcatttgctggaatggtgatcgggaggcttgaaagttcaaaagagcatagcatcactaatggaaagtttacagttggtgtaactatgtttggggttacgactccttgtgttaatgctgtcaaagaaagattagtgaaagaaggatatgagactttggtgttccatgccacgggtgtcgggggcagggccatggaggatcttgttagaggaggttttatacagggtgtgctggatattacgacaactgaggttgcagattacgtagttggaggagtaatggcatgtgatagttcccgatttgatgcaatattagagaagaaaattcctttggttctgagtgtgggagcactggatatggtgaattttggtcctaaaactaccatacctcctgagtttcaacaaagaaagatccatgaacataatgagcaggtttccctaatgcgtactacagtaggtgaaaataagaaatttgctgcatttatagcagaaaagttgaacaaggcatcatcaagtgtatgtgtttgcttgccagagaaaggcgtgtctgcattggatgcacccgggaaagacttttatgatcctgaggcaactagttgtcttacacgtgaactacagatgcttcttgaaaataatgaacgttgtcaggttaaggtcctcccttaccatatcaatgatgcggagtttgcaaatgctttagttgattcattcttggaaatctctccgaaatctagacacgtagaatgtcagccagctgagtccaaatctatccaagacattcagaatgataatgctgttctagagaaatatccctcatgcaacgggaaaaacttttctcgcctgaatgactttccaaatgcaaaaccagaaactttgcagaaaagaactgtgatactgcagaaattgaaagatcaaataagtaagggcaagcctattattggggctggtgctggtacaggtatttctgctaagtttgaggaagctggtggtgtagatttgattgtcttgtacaactcagggcgctttaggatggcaggaaggggatccttagctggtctactgccctttgctgatgcaaatgccattgtacttgagatggccaacgaagtattgcctgtggttaaggaagtggcagttctggctggagtttgtgctactgatcctttccgcaggatggacaacttcctgaagcagttggaatccgttggattctgtggggtgcaaaactttccaactgttggtctgtttgacggtaacttcagacaaaatttggaagagactggaatgggttatggcttggaggttgagatgattgcagcagctcacaggatgggccttttgacaaccccatatgctttctgcccagatgaagcagttgctatggcagaagctggtgccgacatcatagttgctcatatggggcttacaacatctggttcaattggtgcaaaaacagccgtctcattggaggaaagtgtaacttgcgttcaagctattgcagatgctactcataggatatatcctgatgcaattgtgctctgccatggaggccctatatcttcccctgaagaagcagcatatgtactgaagagaaccacaggagttcatggattttatggcgcttcaagcatggaaagactaccagttgagcaagctataactgcaactgtccagcagtacaagtctatttctatggagtgagatgcagtggattttggttctttaaatgtccgaacctatatgtatggtcttcacctctttctttatgtatgatcatgtccagggctcctctgtactcatctagcaacttgaaacactacagcactgctgctgcagtttttatctgtccatatgatatcaatttcaacaagttatatcttttcgtcaaaaaaaaaaaaaaaa

SEQ ID NO: 21 - DNA, 88 bp, Lycopersicon esculentum
  polymorphism (38)..(38): susceptible = G; resistant = A
caaagctcttggaaactttcctaagtataagctaatgrtgaacagaatcttgctggagtagattggccttgggggtagtggaggaaca

SEQ ID NO: 22 - DNA, 48 bp, Lycopersicon esculentum
gaaggtgaccaagttcatgctcaatyactccagcaagattctgttcat

SEQ ID NO: 23 - DNA, 43 bp, Lycopersicon esculentum
gaaggtcggagtcaacggattactccagcaagattctgttcac

SEQ ID NO: 24 - DNA, 26 bp, Lycopersicon esculentum
caaagctctkgaaactttcctaagta

SEQ ID NO: 25 - DNA, 6393 bp, Tomato Brown Rugose Fruit virus
gtatttttgttttacaacatataccaacaacaacaaacaacaaacaacaacattacaattactatttacaactacaatggcatacacacagacagctaccacatccgctttgctcgacactgtccgaggtaacaataccttggtcaacgatcttgcgaagcggcgtctttatgacacagcggtcgacgagttcaacgctcgtgatcgcaggcccaaagtaaatttttccaaagtaataagtgaggaacagacgcttattgctactagggcatatccagaattccagataaccttctataatacgcagaacgccgtgcattcgcttgccggtggactacgatccttagaactggaatatctaatgatgcagatcccgtacggatcactcacatatgatataggtgggaattttgcatctcatctgttcaaaggacgggcatatgttcactgctgtatgcccaatcttgatgtccgcgacataatgcggcacgaaggccagaaagacagtatagaattatacctttccaggcttgagcggggcaacaaagttgtcccaaatttccaaaaggaagcttttgacagatacgctgaaacgccagacgaagttgtctgtcacagtaccttccaaacgtgtacgcaccagcaggtggaaaacacaggcagggtgtatgctattgcattgcacagtatatacgatatacctgctgatgaattcggagcggcacttttaaggaaaaatgtccatgtttgttacgccgccttccacttttccgagaatttacttctcgaagattcacacgtcaaccttgacgaaatcaacgcgtgtttttcgcgtgatggagacaagctgactttttctttcgcatctgagagcactttaaattattgtcatagttattctaatattttaaaatacgtgtgcaaaacttacttcccggcatctaatagagaggtctacatgaaggagtttttggtcaccagggttaacacctggttttgtaagttttctaggatagatacttttttattatacaagggggtagcccacaaaggtgtaaatagtgagcaattttacagcgcaatggaagatgcatggcactacaaaaagactcttgcaatgtgtaacagcgagaggattcttcttgaagattcctcatcggtcaattactggttcccaaaaatgagagatatggtcatagttcctctattcgacatatctctcgacaccagtaaaaggacccgcaaagaagtcttagtgtcaaaggattttgtattcacagttttaaatcacattcgcacttatcaagccaaggcacttacatactccaatgttttatcctttgtcgaatcaattcgttcaagggtaattatcaacggagtgactgccaggtctgagtgggatgttgacaaatctcttttgcaatccttgtccatgacatttttcttgcatactaagcttgccgttttaaaagacgaattgttaatcagcaagtttagtttggggccaaaatcagtaagccagcatgtatgggatgagatttccctggcttttggaaacgcatttccatcgatcaaggagagactgctaaatcggaaactaattaaagtgtcgggagacgcattagaaatcagggtgcctgatttatatgtgacttttcacgatagattagtgactgagtacaaaacatcggtggatatgccagtgcttgatatcagaaagagaatggaggagactgaggttatgtacaatgcattgtctgagctatctgtgctcaaggagtcggacaagttcgacgttgatgttttttcccggatgtgccagactttggaggtagacccaatgactgcagcaaaggttattgtggcagtgatgagcaacgagagcggactgactcttacattcgaacagccaactgaagcaaatgtcgcattggcacttaaagattcagaaaaagcctctgagggtgcactagtggttacttctagagatgttgaagaaccatccatgaagggttcaatggcaagaggagagttacaattggccggtctgtctggagaccaaccagagtcttcctatactcggaacgaggaaatagagtcattagagcaattccacatggcaacggctagttcgttaattcggaaacagatgagttcgattgtgtacacgggccccattaaagttcagcaaatgaaaaactttattgatagcctggtagcatcactctctgctgcggtgtcgaacctagtcaagatcctaaaggatacagctgctatagatctcgaaacccgtcagaagtttggagtcttagatgttgcgaccaaaagatggttaattaaacctttagccaagaatcacgcatggggcgttattgaaacacatgctaggaagtaccacgttgcacttttggagtatgatgagcatggagtggtaacttgcgacagttggagaagggtggccgtgagttctgagtcaatggtttattctgatatggcaaagctcagaacactgaggagattattaagagatggtgagcctcatgtcagcagtgctaaagtcgtcctagttgacggtgtcccgggttgtggaaagacaaaagagattctctcgaaagtaaattttgaggaagatctaatcttagtaccgggtaagcaggctgctgaaatgataaagaggcgtgctaatgcgtcaggaataattcaagccacaagagataatgttcgtactgttgattcatttataatgaattacggtaaaggaacacgctgtcagttcaaaaggttatttatcgacgaaggtctgatgttgcacactggttgtgtgaattttcttgtttctatgtctctgtgcgaaattgcatatgtttatggagacacacaacaaattccatacatcaacagagtatccggttttccgtaccctgcacattttgcaaaaatagaggttgatgaggtggaaactcgcagaactacgctgcgttgtccagccgacattacccactatcttaacagaaggtacgaaggatatgtcatgtgtacatcgtcggttaaaaagtcagtttctcaggaaatggtgagcggggccgcaatgatcaatcctgtatctaagccattgaatgggaaagttttgactttcactcagtctgataaagaggcgctgctttctcgaggatatacggacgtccatacagtacatgaggtacaaggtgagacatatgcagatgtgtcgttggtcagattgactccgacacctgtatctatcatcgcaggagatagtccgcacgttctcgtagctttgtcaaggcatacccaaacattgaagtattacaccgtagtgatggatcctcttgtaagtataattagggatttagaaaaacttagttcttacttgttagatatgtataaagtagatgcagggacccaatagcaattacaggtagactccgtgtttaaaggttctaatctttttgttgcagcaccaaagactggagatatctcagatatgcaattttactatgataagtgtctcccaggtaatagcaccatgttaaataactatgatgctgttaccatgaggttgactgacatttctcttaatgtcaaagattgcatattggatttctctaagtctgtggctgcaccgaaggatccgatcaaaccactgattccgatggtacgaacggcggcagaaatgccacgccagactggactattggaaaatttggtggcgatgatcaaaagaaactttaattcaccggagttatcaggaataatcgacattgagaatactgcatctttagtagtagataaattttttgatagttacttgcttaaagaaaaaagaaaaccaaataaaaatgtttctttattttgtagagagtctctcaatagatggttagagaagcaggagcaagtgaccattggtcagcttgcagattttgattttgtggatcttcctgccgttgatcagtacaggcatatgattaaagcgcaacctaagcagaagctggatacatcaattcaaagcgaatatccggccttgcagacgattgtgtatcattcgaaaaagatcaacgcaatcttcggtcctttgttcagtgagctcacaaggcaaatgctcgaaagcatagactcaagtaagtttttgttctttacaaggaagacgccagctcaaattgaggatttcttcggagatctcgatagccatgtccctatggatatcttggagttggatatttcgaagtatgacaaatctcagaacgagttccactgtgcagtagagtatgaaatatggagaagacttggattagaagattttctgggagaagtttggaaacaaggccacaggaaaactactcttaaagattacacagctggtattaaaacgtgtttatggtaccagagaaagagtggggacgttacaacattcatcggtaatacggtgattattgctgcttgtttagcttccatgttgcccatggagaaaataatcaaaggtgcattttgcggagatgacagtttactatacttcccaaaaggttgtgagtttcctgacatacagcatacagccaaccttatgtggaatttcgaggctaagctattcagaaagcagtatggttatttctgtggaaggtacgtgatacatcatgacagagggtgtattgtttattatgaccctttgaagttgatttctaaacttggtgctaaacacatcaaggattgggatcacttagaagagttcagaagatccctttgtgatgttgcaaattcgttgaacaactgtgcgtattacacgcagttggacgacgctgtgagtgaggtccataaaaccgcacccccgggttcgtttgtatataaaagtttagttaaatatctgtccgataaggttctttttagaagtttgtttatagatggctcttgttaagggtaaagtcaatattaatgagttcatagacttgtcaaaatcagaaaaatttcttccgtctatgttcacacctgttaagagtgtcatgatctccaaggttgataagatattggttcatgaagatgaatctttgtccgaagtcaatttactcaaaggtgtaaaactcattgatggtggctatgtacatcttgctggtcttgtggtgacaggtgaatggaatttgccagataattgtcgtggtggtgtcagtgtctgtttggtcgataagagaatggagagagcggacgaggcaactcttgcttcatactataccgcagcggctaagaaaaggtttcagttcaaagtcgttccaaattacaacatcactaccaaggacgcagaaaaggcagtttggcaagtactagttaatattagaaatgttaaaattgctgcgggttactgtccgctgtcattagaatttgtgtcagtgtgtattgtttataaaaatattataaaactcggtttgagagagaaaattacgagcgtcacggatggagggcccatggaactatcagaagaagttgttgatgagttcatggaagaagtcccgatgtctgtaaggcttgcaaaatttcgttcgaagaccggaaaaaagtttagtagtaaaagtgagaataatagtggtaataataggccgaaaccagacaaaaaccaaaggaaggaaaagggtttaaaagttagggttgagaaggataatttaattgataatgaattggagacttacgtcgccgattcagattcgtattaaatatgtcttacacaatcgcaactccatcgcaatttgtgtttttgtcatcagcatgggccgaccctatagaattaataaatttatgtactaattcactaggtaatcagttccaaacacaacaagctagaacaaccgttcaacggcaatttagcgaagtgtggaaacctgtccctcaagtcactgttaggtttcctgacagtggttttaaggtgtataggtacaatgcggtactagatcctctagttactgctttgttaggagctttcgatactagaaataggattatagaagtcgaaaatcaggcgaacccgacaaccgccgaaacgttagacgctactcgtagagtagatgacgcaacggtggctataaggagcgctataaataatttagtagtagaattggtcaaaggaacaggtttgtacaatcagagcacatttgaaagtgcatccggtttacaatggtcctctgcacctgcatcttgagataatcgagatgcttaaataacagattgtgtctgcaaacacacgtggtacgtacgataacgtatagtgtttttccctccacttaaatcgaagggtagtgtcttggagcgcgcgggacaaatgtgtatggttcatacacatccgtaggcacgtaataaagcgagggattcgaattcccccggaacccccggaggggccca

SEQ ID NO: 26 - DNA, 6392 bp, Tomato Brown Rugose Fruit virus
gtgtattttttacaacatataccaacaacaacaaacaacaaacaacaacattacaattactatttacaactacaatggcatacacacagacagctaccacatccgctttgctcgacactgtccgaggtaacaataccttggtcaacgatcttgcgaagcggcgtctttatgacacagcggtcgacgagttcaacgctcgtgatcgcaggcccaaagtaaatttttccaaagtaataagtgaggaacagacgcttattgctactagggcatatccagaattccagataaccttctataatacgcagaacgccgtgcattcgcttgccggtggactacgatccttagaactggaatatctaatgatgcagatcccgtacggatcactcacatatgatataggtgggaattttgcatctcatctgttcaaaggacgggcatatgttcactgctgtatgcccaatcttgatgtccgcgacataatgcggcacgaaggccagaaagacagtatagaattatacctttccaggcttgagcggggcaacaaagttgtcccaaatttccaaaaggaagcttttgacagatacgctgaaacgccagacgaagttgtctgtcacagtaccttccaaacgtgtacgcaccagcaggtggaaaacacaggcagggtgtatgctattgcattgcacagtatatacgatatacctgctgatgaattcggagcggcacttttaaggaaaaatgtccatgtttgttacgccgccttccacttttccgagaatttacttctcgaagattcacacgtcaaccttgacgaaatcaacgcgtgtttttcgcgtgatggagacaagctgactttttctttcgcatctgagagcactttaaattattgtcatagttattctaatattttaaaatacgtgtgcaaaacttacttcccggcatctaatagagaggtctacatgaaggagtttttggtcaccagggttaacacctggttttgtaagttttctaggatagatacttttttattatacaagggggtagcccacaaaggtgtaaatagtgagcaattttacagcgcaatggaagatgcatggcactacaaaaagactcttgcaatgtgtaacagcgagaggattcttcttgaagattcctcatcggtcaattactggttcccaaaaatgagagatatggtcatagttcctctattcgacatatctctcgacaccagtaaaaggacccgcaaagaagtcttagtgtcaaaggattttgtattcacagttttaaatcacattcgcacttatcaagccaaggcacttacatactccaatgttttatcctttgtcgaatcaattcgttcaagggtaattatcaacggagtgactgccaggtctgagtgggatgttgacaaatctcttttgcaatccttgtccatgacatttttcttgcatactaagcttgccgttttaaaagacgaattgttaatcagcaagtttagtttggggccaaaatcagtaagccagcatgtatgggatgagatttccctggcttttggaaacgcatttccatcgatcaaggagagactgctaaatcggaaactaattaaagtgtcgggagacgcattagaaatcagggtgcctgatttatatgtgacttttcacgatagattagtgactgagtacaaaacatcggtggatatgccagtgcttgatatcagaaagagaatggaggagactgaggttatgtacaatgcattgtctgagctatctgtgctcaaggagtcggacaagttcgacgttgatgttttttcccggatgtgccagactttggaggtagacccaatgactgcagcaaaggttattgtggcagtgatgagcaacgagagcggactgactcttacattcgaacagccaactgaagcaaatgtcgcattggcacttaaagattcagaaaaagcctctgagggtgcactagtggttacttctagagatgttgaagaaccatccatgaagggttcaatggcaagaggagagttacaattggccggtctgtctggagaccaaccagagtcttcctatactcggaacgaggaaatagagtcattagagcaattccacatggcaacggctagttcgttaattcggaaacagatgagttcgattgtgtacacgggccccattaaagttcagcaaatgaaaaactttattgatagcctggtagcatcactctctgctgcggtgtcgaacctagtcaagatcctaaaggatacagctgctatagatctcgaaacccgtcagaagtttggagtcttagatgttgcgaccaaaagatggttaattaaacctttagccaagaatcacgcatggggcgttattgaaacacatgctaggaagtaccacgttgcacttttggagtatgatgagcatggagtggtaacttgcgacagttggagaagggtggccgtgagttctgagtcaatggtttattctgatatggcaaagctcagaacactgaggagattattaagagatggagagcctcatgtcagcagtgctaaagtcgtcctagttgacggtgtcccgggttgtggaaagacaaaagagattctctcgaaagtaaattttgaggaagatctaatcttagtaccgggtaagcaggctgctgaaatgataaagaggcgtgctaatgcgtcaggaataattcaagccacaagagataatgttcgtactgttgattcatttataatgaattacggtaaaggaacacgctgtcagttcaaaaggttatttatcgacgaaggtctgatgttgcacactggttgtgtgaattttcttgtttctatgtctctgtgcgaaattgcatatgtttatggagacacacaacaaattccatacatcaacagagtatccggttttccgtaccctgcacattttgcaaaaatagaggttgatgaggtggaaactcgcagaactacgctgcgttgtccagccgacattacccactatcttaacagaaggtacgaaggacatgtcatgtgtacatcgtcggttaaaaagtcagtttctcaggaaatggtgagcggggccgcaatgatcaatcctgtatctaagccattgaatgggaaagttttgactttcactcagtctgataaagaggcgctgctttctcgaggatatacggacgtccatacagtacatgaggtacaaggtgagacatatgcagatgtgtcgttggtcagattgactccgacacctgtatctatcatcgcaggagatagtccgcacgttctcgtagctttgtcaaggcatacccaaacattgaagtattacaccgtagtgatggatcctcttgtaagtataattagggatttagaaaaacttagttcttacttgttagatatgtataaagtagatgcagggacccaatagcaattacaggtagactccgtgtttaaaggttctaatctttttgttgcagcaccaaagactggagatatctcagatatgcaattttactatgataagtgtctcccaggtaatagcaccatgttaaataactatgatgctgttaccatgaggttgactgacatttctcttaatgtcaaagattgcatattggatttctctaagtctgtggctgcaccgaaggatccgatcaaaccactgattccaatggtacgaacggcggcagaaatgccacgccagactggactattggaaaatttggtggcgatgatcaaaagaaactttaattcaccggagttatcaggaataatcgacattgagaatactgcatctttagtagtagataaattttttgatagttacttgcttaaagaaaaaagaaaaccaaataaaaatgtttctttattttgtagagagtctctcaatagatggttagagaagcaggagcaagtgaccattggtcagcttgcagattttgattttgtggatcttcctgccgttgatcagtacaggcatatgattaaagcgcaacctaagcagaagctggatacatcaattcaaagcgaatatccggccttgcagacgattgtgtatcattcgaaaaagatcaacgcaatcttcggtcctttgttcagtgagctcacaaggcaaatgctcgaaagcatagactcaagtaagtttttgttctttacaaggaagacgccagctcaaattgaggatttcttcggagatctcgatagccatgtccctatggatatcttggagttggatatttcgaagtatgacaaatctcagaacgagttccactgtgcagtagagtatgaaatatggagaagacttggattagaagattttctgggagaagtttggaaacaaggccacaggaaaactactcttaaagattacacagctggtattaaaacgtgtttatggtaccagagaaagagtggggacgttacaacattcatcggtaatacggtgattattgctgcttgtttagcttccatgttgcccatggagaaaataatcaaaggtgcattttgcggagatgacagtttactatacttcccaaaaggttgtgagtttcctgacatacagcatacagccaaccttatgtggaatttcgaggctaagctattcagaaagcagtatggttatttctgtggaaggtacgtgatacatcatgacagagggtgtattgtttattatgaccctttgaagttgatttctaaacttggtgctaaacacatcaaggattgggatcacttagaagagttcagaagatccctttgtgatgttgcaaattcgttgaacaactgtgcgtattacacgcagttggacgacgctgtgagtgaggtccataaaaccgcacccccgggttcgtttgtatataaaagtttagttaaatatctgtccgataaggttctttttagaagtttgtttatagatggctcttgttaagggtaaagtcaatattaatgagttcatagacttgtcaaaatcagaaaaatttcttccgtctatgttcacacctgttaagagtgtcatgatctccaaggttgataagatattggttcatgaagatgaatctttgtccgaagtcaatttactcaaaggtgtaaaactcattgatggtggctatgtacatcttgctggtcttgtggtgacaggtgaatggaatttgccagataattgtcgtggtggtgtcagtgtctgtttggtcgataagagaatggagagagcggacgaggcaactcttgcttcatactataccgcagcggctaagaaaaggtttcagttcaaagtcgttccaaattacaacatcactaccaaggacgcagaaaaggcagtttggcaagtactagttaatattagaaatgttaaaattgctgcgggttactgtccgctgtcattagaatttgtgtcagtgtgtattgtttataaaaatattataaaactcggtttgagagagaaaattacgagcgtcacggatggagggcccatggaactatcagaagaagttgttgatgagttcatggaagaagtcccgatgtctgtaaggcttgcaaaatttcgttcgaagaccggaaaaaagtttagtagtaaaagtgagaataatagtggtaataataggccgaaaccaaacaaaaaccaaaggaaggaaaagggtttaaaagttagggttgagaaggataatttaattgataatgaattggagacttacgtcgccgattcagattcgtattaaatatgtcttacacaatcgcaactccatcgcaatttgtgtttttgtcatcagcatgggccgaccctatagaattaataaatttatgtactaattcactaggtaatcagttccaaacacaacaagctagaacaaccgttcaacggcaatttagcgaagtgtggaaacctgtccctcaagtcactgttaggtttcctgacagtggttttaaggtgtataggtacaatgcggtactagatcctctagttactgctttgttaggagctttcgatactagaaataggattatagaagtcgaaaatcaggcgaacccgacaaccgccgaaacgttagacgctactcgtagagtagatgacgcaacggtggctataaggagcgctataaataatttagtagtagaattggtcaaaggaacaggtttgtacaatcagagcacatttgaaagtgcatccggtttacaatggtcctctgcacctgcatcttgagataatcgagatgcttaaataacagattgtgtctgcaaacacacgtggtacgtacgataacgtatagtgtttttccctccacttaaatcgaagggtagtgtcttggagcgcgcgggacaaatgtgtatggttcatacacatccgtaggcacgtaataaagcgagggattcgaattcccccggaacccccggtaggggccca

(19) United States
(12) Patent Application Publication
(10) Pub. No.: US 2022/0304272 A1
(43) Pub. Date: Sep. 29, 2022

(54) RESISTANCE IN PLANTS OF SOLANUM LYCOPERSICUM TO THE TOBAMOVIRUS TOMATO BROWN RUGOSE FRUIT VIRUS

(71) Applicant: Vilmorin & Cie, Paris (FR)

(72) Inventors: Varda Ashkenazi, Berurim (IL); Yaniv Rotem, Berurim (IL); Ron Ecker, Berurim (IL); Shai Nashilevitz, Berurim (IL); Naama Barom, Berurim (IL)

(73) Assignee: Vilmorin & Cie, Paris (FR)

(21) Appl. No.: 17/619,176

(22) PCT Filed: Jun. 12, 2020
(86) PCT No.: PCT/EP2020/066398

§ 371 Date: Dec. 14, 2021

(30) Foreign Application Priority Data
Jun. 14, 2019 (IB) PCT/IB2019/000674

(51) Int. Cl. A01H 6/82; A01H 5/08

(52) U.S. Cl. CPC A01H 6/825; A01H 5/08

(57) ABSTRACT

The invention relates to a Solanum lycopersicum plant resistant to Tomato Brown Rugose Fruit virus comprising in its genome the combination of the Tm-1 resistance gene on chromosome 2, and at least one quantitative trait locus (QTL) chosen from QTL3 on chromosome 11, QTL1 on chromosome 6 and QTL2 on chromosome 9, that independently confer to the plant foliar and/or fruit tolerance to TBRFV, wherein said QTLs are present in the genome of a plant of the seeds HAZTBRFVRES1 NCIMB accession number 42758. The combination of at least one of these QTLs with the Tm-1 gene delays, reduces or inhibits the replication or multiplication of the virus in the plants of the invention. The invention is also directed to parts of these plants with TBRFV resistance phenotype, as well as progeny, to the use of these plants for introgressing the resistance in another genetic background, as well as to different methods for obtaining tomato plants or seeds with increased resistance to Tomato Brown Rugose Fruit virus.

The present invention relates to resistance in plants of Solanum lycopersicum, also known as 5 Lycopersicum esculentum, to the tobamovirus Tomato Brown Rugose Fruit virus (TBRFV, also known as ToBRFV). More specifically, the present invention relates to tomato plants and fruits comprising one or more genetic determinants, in combination with the Tm-1 resistance gene, that lead to resistance to the Tomato Brown Rugose Fruit virus. The invention further relates to markers linked to these one or more genetic determinant(s) and Tm-1 gene and to the use of such markers to identify or select plants carrying such resistance. The invention also relates to the seeds and progeny of such plants and to propagation material for obtaining such plants, and to different uses of these plants.

### BACKGROUND OF THE INVENTION

All cultivated and commercial forms of tomato belong to a species most frequently referred to as Lycopersicon esculentum Miller. Lycopersicon is a relatively small genus within the extremely large and diverse family Solanaceae which is considered to consist of around 90 genera, including pepper, tobacco and eggplant. The genus Lycopersicon has been divided into two subgenera, the esculentum complex which contains those species that can easily be crossed with the commercial tomato and the peruvianum complex which contains those species which are crossed with considerable difficulty (Stevens, M., and Rick, C. M. 1986). Due to its value as a crop, L. esculentum Miller has become widely disseminated all over the world. Even if the precise origin of the cultivated tomato is still somewhat unclear, it seems to come from the Americas, being native to Ecuador, Peru and the Galapagos Island and initially cultivated by Aztecs and Incas as early as 700 AD. Mexico appears to have been the site of domestication and the source of the earliest introduction. It is supposed that the cherry tomato, L. esculentum var. cerasiforme, is the direct ancestor of modern cultivated forms.

Tomato is grown for its fruit, widely used as a fresh market or processed product. As a crop, tomato is grown commercially wherever environmental conditions permit the production of an economically viable yield. The majority of fresh market tomatoes are harvested by hand at vine ripe and mature green stage of ripeness. Fresh market tomatoes are available year round. Processing tomato are mostly mechanically harvested and used in many forms, as canned tomatoes, tomato juice, tomato sauce, puree, paste or even catsup.

Tomato is a normally simple diploid species with twelve pairs of differentiated chromosomes. However, polyploidy tomato is also part of the present invention. The cultivated tomato is self-fertile and almost exclusively self-pollinating. The tomato flowers are hermaphrodites. Commercial cultivars were initially open pollinated. As hybrid vigor has been identified in tomatoes, hybrids are replacing the open pollinated varieties by gaining more and more popularity amongst farmers with better yield and uniformity of plant characteristics. Due to its wide dissemination and high value, tomato has been intensively bred. This explains why such a wide array of tomato is now available. The shape may range from small to large, and there are cherry, plum, pear, blocky, round, and beefsteak types. Tomatoes may be grouped by the amount of time it takes for the plants to mature fruit for harvest and, in general the cultivars are considered to be early, midseason or late-maturing. Tomatoes can also be grouped by the plants growth habit; determinate, semi-determinate or indeterminate. Determinate plants tend to grow their foliage first, then set flowers that mature into fruit if pollination is successful. All of the fruits tend to ripen on a plant at about the same time. Indeterminate tomatoes start out by growing some foliage, then continue to produce foliage and flowers throughout the growing season. These plants will tend to have tomato fruit in different stages of maturity at any given time. The semi-determinate tomatoes have a phenotype between determinate and indeterminate, they are typical determinate types except that grow larger than determinate varieties. More recent developments in tomato breeding have led to a wider array of fruit color. In addition to the standard red ripe color, tomatoes can be creamy white, lime green, pink, yellow, golden, orange or purple. Hybrid commercial tomato seed can be produced by hand pollination. Pollen of the male parent is harvested and manually applied to the stigmatic surface of the female inbred. Prior to and after hand pollination, flowers are covered so that insects do not bring foreign pollen and create a mix or impurity. Flowers are tagged to identify pollinated fruit from which seed will be harvested.

A variety of pathogens affect the productivity of tomato plants, including virus, fungi, bacteria, nematodes and insects. Tomatoes are inter alia susceptible to many viruses and virus resistance is therefore of major agricultural importance.

Tobamoviruses are among the most important plant viruses causing severe damages in agriculture, especially to vegetable and ornamental crops around the world. Tobamoviruses are easily transmitted by mechanical means while there is no evidence of a natural vector, as well as through seed transmission. Tobamoviruses are generally characterized by a rod-shaped particle of about 300 nm, their structure consists in a single stranded, positive RNA genome encoding four proteins, encapsidated by 17 KDa coat protein (CP) molecules.

In tomatoes, tobacco mosaic virus (TMV), tomato mosaic virus (ToMV) are feared by growers worldwide as they can severely damage crop production, for example through irregular ripening (fruits having yellowish patches on the surface and brownish spots beneath the surface). Several genes have however been identified by plants breeders over the years.

The first resistance gene identified was the Tm-1 gene, conferring resistance to TMV. This gene, introgressed from S. habrochaites, is incompletely dominant and homozygosis was generally required for the TMV resistance. The Tm-1 gene was however overcome within about one year of introduction to commercial horticulture, rendering the pursuing of its introduction into other commercial lines entirely useless (Pelham et al, 1970. “The establishment of a new strain of tobacco mosaic virus resulting from the use of resistant varieties of tomato”; Ann. Appl. Biol., 65:293-297). This gene was also identified as conferring resistance to ToMV, but today, the vast majority of the circulating TMV and ToMV strains are able to infect commercial plants harboring the Tm-1 gene, such that this gene is no longer considered as a resistance gene against TMV/ToMV infection in commercial plants. The use of this Tm-1 gene has now been almost completely abandoned in favor of alternative resistance genes.

For the last decades, all modern indeterminate tomato varieties and many of the determinate tomato varieties indeed contain the Tm-2 gene or preferably the Tm-2² allele of this gene, which give them immunity to almost all known races of Tobamoviruses which affected commercial tomatoes (ToMV and TMV) before 2014.

During 2014-2015, a severe outbreak of virus affected tomato productions areas in the middle east, such as in Jordan and in Israel. Most of the tomato varieties affected were considered TMV and/or ToMV resistant, but were still severely affected and showed typical TMV/ToMV like symptoms: while the foliar ones were quite similar to the TMV/ToMV symptoms, the fruit symptoms were much more frequent and severe than the usual symptoms from such viruses with fruits lesions and deformations. The fruit quality was very poor and rather unmarketable. Salem et al (Arch. Virol. 161 (2), 503-506. 2015) extracted RNA from fruit and leaves of symptomatic plants, infected in Jordan, and made various tests leading to the identification of a new Tobamovirus species, the sequence of which corresponds to GenBank accession no. KT383474 (SEQ ID No:25); Salem et al proposed to name this Jordanian virus: Tomato Brown Rugose Fruit virus (TBRFV or ToBRFV). The comparison to other Tobamoviruses sequences showed that it is indeed a Tobamovirus, but not TMV or ToMV. The resistance to TMV and/or ToMV does not confer resistance to this new virus TBRFV.

Luria et al (PLoS One. 2017; 12(1): e0170429) have concomitantly isolated and sequenced the complete genome of the Israeli tobamovirus infecting tomato in Israel, corresponding to GenBank accession no. KX619418 (SEQ ID No:26). They have thus shown a very high sequence identity between the Israeli and the Jordanian viruses (more than 99% sequence identity) and have concluded to two different isolates of tomato brown rugose fruit virus.

Recently, the virus was identified in Europe, especially in Sicily, Germany and the Netherlands, and in Mexico, and therefore now it is considered as a major global threat to tomato crop. The strain identified appears to be the Israeli strain, rather than the Jordanian strain.

In Israel, the present inventors collected isolates, and 7 representative isolates from all crop production areas (North, Center and South) were sequenced. Sequence comparison to the sequence of the Jordanian ToBRFV seems to indicate that all the Israeli isolates are essentially but not entirely identical to the Jordanian isolate, thus confirming they are probably to be considered as two different strains of the same virus in both countries.

In a previous application, the present inventors have first identified tomato plants which display tolerance to the Tomato Brown Rugose Fruit virus and they have been able to localize and identify genetic determinants, also referred to hereafter as QTLs (Quantitative Trait Locus) that lead to tolerance to the Tomato Brown Rugose Fruit virus. Two QTLs, namely QTL1 and QTL2, are to be found on chromosome 6 and 9 respectively, and confer independently or in combination an improved tolerance in the fruits of a tomato plant infected or likely to be infected by the TBRFV, when present homozygously into a S. lycopersicum background. A third QTL, QTL3, is to be found on chromosome 11, and confers an improved tolerance in the leaves of a tomato plant infected or likely to be infected by the TBRFV, when present homozygously. These QTLs are those referred to and described in PCT application WO2018/219941. These QTLs will be called tolerance QTLs in the following description.

Whereas these QTLs, either alone or in combination, provide tolerance to TBRFV, the inventors have now established that, most of the time, they cannot confer resistance to the tomato plants, especially they cannot confer a level of resistance sufficient to delay, reduce or inhibit the replication or multiplication of the virus. Indeed, infected plants bearing one or more of said tolerance QTLs are nevertheless propagating the virus, which remains a threat for all surrounding tomato plants not bearing these QTLs.

As Tobamoviruses are not easily controlled but through genetic improvement by the identification and use in breeding of resistance genes, and as the resistance genes currently available to control TMV and/or ToMV are useless against the damages and propagation from the new Tomato Brown Rugose Fruit virus, and the tolerance QTLs not able to stop or sufficiently reduce the viral propagation, there is an urgent need to identify resistance against this new Tobamovirus, failing that would result in entire regions in which tomato crop could not be produced anymore.

### SUMMARY

The present inventors have identified tomato plants which display resistance to the Tomato Brown Rugose Fruit virus and they have been able to identify the combination of genetic determinants that leads to the resistance to the Tomato Brown Rugose Fruit virus, namely the combination of QTLs (Quantitative Trait Locus) and gene providing this resistance or enhanced tolerance.

The resistance according to the present invention is imparted by the Tm-1 resistance gene, when combined with genetic determinants or QTLs, wherein these QTLs confer only tolerance to the Tomato Brown Rugose Fruit virus (TBRFV) at the level of the leaves and/or the fruits of the tomato plants, when they are not combined with the Tm-1 resistance gene. These QTLs or genetic determinants are described as being of recessive nature, according to WO2018/219941. The presence of the Tm-1 resistance gene at the homozygous state is not necessary, contrary to the main mode of action of the Tm-1 gene with regard to past resistance to TMV/ToMV, although this resistance has now been overcome by the circulating strains of TMV/ToMV.

The fruit tolerance is imparted independently by QTL1 or QTL2, and the foliar tolerance by QTL3, their transfer to different genetic background, i.e. into various tomatoes can be easily carried out by a skilled artisan in plant breeding, especially given the information regarding suitable markers associated with the QTLs provided in WO2018/219941. The same is also true for the Tm-1 gene. The present invention thus provides the combination of:
genetic determinants, also named here QTLs or tolerance QTLs, conferring, when present in the homozygous state, the phenotype of TBRFV tolerance at the level of the tomato leaves and/or fruits of the tomato plants infected by the TBRFV, and
the Tm-1 gene,
   wherein this combination provides resistance to the TBRFV, in particular the ability to delay, reduce and/or inhibit the replication of the virus, whereas neither the QTLs alone or in combination, nor the Tm-1 gene alone, provides such a level of resistance or of enhanced tolerance.

The present invention also concerns commercial S. lycopersicum plants that display resistance to TBRFV as well as methods that produce or identify S. lycopersicum plants or populations (germplasm) that display resistance to TBRFV. The present invention also discloses molecular genetic markers, especially SNPs, linked to the tolerance QTLs and to the Tm-1 gene, which can be used in any selection method for obtaining the plant of the invention. Plants obtained through the methods and uses of such molecular markers are also provided.

The invention also provides several methods for improving the yield of tomato production in an environment infested by TBRFV and methods for protecting a tomato field from TBRFV infestation.

### Definitions

The term “Resistance” is as defined by the ISF (International Seed Federation) Vegetable and Ornamental Crops Section for describing the reaction of plants to pests or pathogens, and abiotic stresses for the Vegetable Seed Industry. Specifically, by resistance, it is meant the ability of a plant variety to restrict the growth and development of a specified pest or pathogen and/or the damage they cause when compared to susceptible plant varieties under similar environmental conditions and pest or pathogen pressure. Resistant varieties may exhibit some disease symptoms or damage under heavy pest or pathogen pressure.

The term ‘Tolerance’ is used herein to indicate a phenotype of a plant wherein at least some of the disease-symptoms remain absent upon exposure of said plant to an infective dose of virus, whereby the presence of a systemic or local infection, virus multiplication, at least the presence of viral genomic sequences in cells of said plant and/or genomic integration thereof can be established, at least under some culture conditions. Tolerant plants are therefore resistant for symptom expression but symptomless carriers of the virus. Sometimes, viral sequences may be present or even multiply in plants without causing disease symptoms. It is to be understood that a tolerant plant, although it is infected by the virus, is generally able to restrict at least moderately the growth and development of the virus.

In case of TBRFV, by leave tolerance, or foliar tolerance, it is meant the phenotype of a plant wherein the disease symptoms on the leaves remain absent upon exposure of said plant to an infective dose of TBRFV. Disease symptoms on the fruits may however be present on infected plants. By fruit tolerance, in case of TBRFV, it is meant the phenotype of a plant wherein the disease symptoms on the fruits remain absent upon exposure of said plant to an infective dose of TBRFV. Disease symptoms on the leaves may however be present on infected plants.

Symptoms on leaves of TBRFV infection generally include mosaic, distortion of the leaflets and in many cases also shoestrings like symptoms. Symptoms on fruits of TBRFV infection generally include typical yellow lesions and deformation of the fruits. In many cases there are also “chocolate spots” on the fruits.

Susceptibility: The inability of a plant variety to restrict the growth and development of a specified pest or pathogen; a susceptible plant displays the detrimental symptoms linked to the virus infection, namely the foliar damages and fruit damages in case of TBRFV infection.

A S. lycopersicum plant susceptible to Tomato Brown Rugose Fruit virus, is for example the commercially available variety Candela as mentioned in the 2015 Salem et al. publication. It can also be the Hazera No 2 and Hazera No 4 lines mentioned in the PCT application WO2018/219941. All commercially available varieties of tomato grown in TBRFV infected area are, to date, i.e. before the present invention, susceptible to TBRFV, or at best tolerant for those plants bearing the tolerance QTLs, such as the deposited seeds of HAZTBRFVRES1. A sample of this S. lycopersicum seed has been deposited by Hazera Seeds Ltd. Berurim, M. P. Shimim 79837, Israel, pursuant to and in satisfaction of the requirements of the Budapest treaty on the International Recognition of the deposit of Microorganisms for the Purpose of Patent procedure (“the Budapest Treaty” with the National collection of Industrial, Food and Marine bacteria (NCIMB) (NCIMB, Ltd, Ferguson Building, Craibstone Estate, Bucksburn, Aberdeen AB21 9YA, united Kingdom) on 16 May 2017 under accession number 42758.

A plant according to the invention has thus at least improved resistance or increased tolerance to Tomato Brown Rugose Fruit virus, with respect to the variety Candela, and more generally with respect to any commercial variety of tomato grown in Tomato Brown Rugose Fruit virus infected area, including tolerant plant, and with respect to HAZTBRFVRES1.

As used herein, the term “offspring” or “progeny” refers to any plant resulting as progeny from a vegetative or sexual reproduction from one or more parent plants or descendants thereof. For instance an offspring plant may be obtained by cloning or selfing of a parent plant or by crossing two parents plants and include selfings as well as the F1 or F2 or still further generations. An F1 is a first-generation offspring produced from parents at least one of which is used for the first time as donor of a trait, while offspring of second generation (F2) or subsequent generations (F3, F4, etc.) are specimens produced from selfings of F1's, F2's etc. An F1 may thus be (and usually is) a hybrid resulting from a cross between two true breeding parents (true-breeding is homozygous for a trait), while an F2 may be (and usually is) an offspring resulting from self-pollination of said F1 hybrids.

As used herein, the term “cross”, “crossing”, “cross pollination” or “cross-breeding” refer to the process by which the pollen of one flower on one plant is applied (artificially or naturally) to the ovule (stigma) of a flower on another plant.

As used herein, the term “genetic determinant” and/or “QTL” refers to any segment of DNA associated with a biological function. Thus, QTLs and/or genetic determinants include, but are not limited to, genes, coding sequences and/or the regulatory sequences required for their expression. QTLs and/or genetic determinants can also include nonexpressed DNA segments that, for example, form recognition sequences for other proteins.

As used herein, the term “genotype” refers to the genetic makeup of an individual cell, cell culture, tissue, organism (e.g., a plant), or group of organisms.

As used herein, the term “grafting” is the operation by which a rootstock is grafted with a scion. The primary motive for grafting is to avoid damages by soil-born pest and pathogens when genetic or chemical approaches for disease management are not available. Grafting a susceptible scion onto a resistant rootstock can provide a resistant cultivar without the need to breed the resistance into the cultivar. In addition, grafting may enhance tolerance to abiotic stress, increase yield and result in more efficient water and nutrient uses.

As used herein, the term “heterozygote” refers to a diploid or polyploid individual cell or plant having different alleles (forms of a given gene, genetic determinant or sequences) present at least at one locus.

As used herein, the term “heterozygous” refers to the presence of different alleles (forms of a given gene, genetic determinant or sequences) at a particular locus.

As used herein, “homologous chromosomes”, or “homologs” (or homologues), refer to a set of one maternal and one paternal chromosomes that pair up with each other during meiosis. These copies have the same genes in the same loci and the same centromere location.

As used herein, the term “homozygote” refers to an individual cell or plant having the same alleles at one or more loci on all homologous chromosomes.

As used herein, the term “homozygous” refers to the presence of identical alleles at one or more loci in homologous chromosomal segments.

As used herein, the term “hybrid” refers to any individual cell, tissue or plant resulting from a cross between parents that differ in one or more genes.

As used herein, the term “locus” (plural: “loci”) refers to any site that has been defined genetically, this can be a single position (nucleotide) or a chromosomal region. A locus may be a gene, a genetic determinant, or part of a gene, or a DNA sequence, and may be occupied by different sequences. A locus may also be defined by a SNP (Single Nucleotide Polymorphism), by several SNPs, or by two flanking SNPs.

As used herein, the term “rootstock” is the lower part of a plant capable of receiving a scion in a grafting process.

As used herein, the term “scion” is the higher part of a plant capable of being grafted onto a rootstock in a grafting process.

### DETAILED DESCRIPTION OF THE INVENTION

The present inventors have demonstrated that the three QTLs disclosed in WO2018/219941, which, when present homozygously in a S. lycopersicum plant, alone or in combination, provide an improved tolerance in the fruits and/or leaves of a tomato plant infected or likely to be infected by the Tomato Brown Rugose Fruit virus (TBRFV or ToBRFV in the following), are nevertheless not entirely able to restrict the propagation of the virus. Indeed, they have discovered that multiplication of the virus generally occurs within these plants, as evidenced by the detection of viral genomic sequences in cells of said plants. Such plants are thus bearing the virus and are unable to limit the propagation of the virus from plants to plants.

The three QTLs disclosed in WO2018/219941, namely QTL1, QTL2 and QTL3, on chromosomes 6, 9 and 11 respectively, will be referred to in the following as the “tolerance QTLs”. More specifically, the QTL1 and QTL2 will be referred to as the “fruit tolerance QTLs” and QTL3 on chromosome 11 as the “foliar tolerance QTL”.

The present inventors have unexpectedly found that the combination of at least one of said tolerance QTLs, namely QTL1, QTL2 and/or QTL3, with the Tm-1 resistance gene, confers an improved tolerance or resistance in tomato, against TBRFV, especially against the Israeli isolate strain, reduces the virus titer in the tomato plants and/or inhibits the propagation of the virus, and/or delays the progression of the virus and thus of any associated symptoms, and/or enhances the level of resistance. In particular, the resistance is improved or enhanced with respect to the corresponding plant lacking the Tm-1 resistance gene, regarding at least one of the criteria comprising virus titer, virus progression, foliar symptoms of infection or fruit symptoms of infection.

According to a preferred embodiment, at least one of the tolerance QTL is present homozygously, e.g. QTL3, especially if there is only one QTL.

Moreover, it is also preferred that at least two tolerance QTLs are combined with the Tm-1 gene; in such a case, at least one QTL is advantageously present heterozygously, e.g. QTL2. According to an embodiment, there are 2 or 3 of the tolerance QTLs, in combination with the Tm-1 gene, and at least one QTL is present homozygously and at least another one heterozygously.

It is noted that the Tm-1 resistance gene, although previously identified as a resistance gene against TMV and ToMV is no longer providing resistance to circulating ToMV/TMV strains, as the circulating ToMV and TMV strains have mutated in order to escape this resistance. The presence of the Tm-1 resistance gene in the plants of the invention therefore does not provide ToMV and/or TMV resistance to these plants, especially for commercial plants threatened specifically by the circulating ToMV/TMV strains.

As demonstrated in the examples, the phenotype of the plants according to the invention is resistance to TBRFV, namely foliar and/or fruit resistance, and the plants of the invention are capable of improved restriction of the viral propagation, at least at some stages after infection.

By improved restriction of the viral propagation, it is meant that the level of viral sequences (for example as detected by q-RT-PCR) or protein detected in a plant, as measured by ELISA technique at around 70-90 days post inoculation (DPI), is at least 50% inferior to the level of viral sequences detected in a susceptible plant or in a tolerant but not resistant plant, at the same time by the same technique, preferably at least 60% inferior, at least 70% or at least 80% inferior. The level of viral sequences or proteins may also be measured at around 30 DPI; the level is decreased if it is at least 20% inferior to the level measured in a susceptible plant.

According to a first aspect, the invention is thus directed to a Solanum lycopersicum plant, resistant to Tomato Brown Rugose Fruit virus (TBRFV), comprising in its genome the combination of:
the Tm-1 resistance gene, homozygously or heterozygously, and
at least one tolerance quantitative trait locus (QTL), present either homozygously or heterozygously.

Preferably, there are at least two QTLs, preferably one present homozygously and another one present heterozygously.

The invention is also directed to a cell of such plants, as well as seeds comprising said QTLs in combination with a Tm-1 gene.

The tolerance QTL is to be chosen in the group consisting of QTL3 on chromosome 11, QTL1 on chromosome 6 and QTL2 on chromosome 9. Each one of these tolerance QTLs, independently, confers to the plant foliar and/or fruit tolerance to TBRFV, and confers resistance or enhanced tolerance to TBRFV when combined with the Tm-1 gene. Said tolerance QTLs are present in the genome of a plant of the seeds HAZTBRFVRES1 NCIMB accession number 42758.

The Tm-1 gene is as defined inter alia in the publication Ishibashi et al, 2007 (An inhibitor of viral RNA replication is encoded by a plant resistance gene. PNAS Aug. 21, 2007 104 (34) 13833-13838); preferably ‘Tm-1 gene’ refers to a genetic sequence encoding a protein having the Tm-1 activity reported in the article, namely the ability to inhibit the viral replication of a wild-type ToMV strain Tm-1 sensitive, for example the strain ToMV-L disclosed in this article. According to a preferred embodiment, the Tm-1 gene according to the invention is a gene encoding a protein having the 754 amino acid sequence reported in Ishibashi et al, corresponding to SEQ ID No:19 (NCBI BAF75724), or a protein having at least 75%, preferably at least 80%, more preferably at least 85%, 90%, or 95% sequence identity with SEQ ID No:19 and exhibiting the Tm-1 activity reported in Ishibashi et al, 2007, namely the ability to inhibit viral RNA replication of a wild-type Tm-1 sensitive ToMV strain. According to a preferred embodiment, this gene has a sequence corresponding to the mRNA sequence referred to in Ishibashi et al, 2007, namely sequence NCIB AB287296 (SEQ ID No:20), or a sequence having at least 50%, preferably at least 60%, at least 70%, more preferably at least 75%, 80%, 85%, 90%, or 95% sequence identity with SEQ ID No:20. Irrespective of the degree of sequence identity with SEQ ID No:20, a Tm-1 gene according to the invention preferably encodes a protein exhibiting the Tm-1 activity reported in Ishibashi et al, 2007, namely the ability to inhibit viral RNA replication of wild-type ToMV.

It is preferred that, in the genome of a plant, seed or cell of the invention, the Tm-1 gene be present on chromosome 2. The present invention however also encompasses plant, seed or cell, comprising the Tm-1 gene at a locus which does not correspond to the locus mentioned in Ishibashi et al, 2007.

The invention thus encompasses S. lycopersicum plants, cells or seeds, comprising in their genome various combinations of QTL1, QTL2 and QTL3, preferably at least one QTL being at the homozygous state and/or at least one being at the heterozygous state, in association with Tm-1 gene. Preferably, there are at least 2 QTLs, and at least one is at the homozygous state and at least one at the heterozygous state. The invention thus encompasses plants comprising the combination of QTL3 and Tm-1, the combination of QTL1 and Tm-1, the combination of QTL2 and Tm-1, the combination of QTL3, QTL1 and Tm-1, the combination of QTL3, QTL2 and Tm-1, the combination of QTL1, QTL2 and Tm-1 and the combination of QTL1, QTL2, QTL3 and Tm-1. Particularly preferred combinations are QTL3 and Tm-1, and QTL2, QTL3 and Tm-1. Different alternative combinations are disclosed in Table 1 below; it is particularly preferred that QTL3 be present at the homozygous state and QTL2 at the heterozygous state. The Tm-1 resistance gene can be found heterozygously or homozygously. A preferred combination is for example QTL3 at the homozygous or heterozygous state, QTL2 at the heterozygous state and Tm-1 homozygously.

It is to be understood that, in the context of the present invention, preferably at least one of the tolerance QTL is to be found homozygously in the genome of the plants, whereas the Tm-1 resistance gene can be found heterozygously or homozygously.

According to an embodiment, the QTL2 on chromosome 9 is present heterozygously in a plant according to the invention.

In another preferred embodiment, the plant comprises homozygously QTL3, in combination with Tm-1, either homozygously or heterozygously. Such a plant may advantageously also comprise QTL2, preferably heterozygously.

According to a preferred embodiment, the Tm-1 resistance gene is also to be found at the homozygous state. In a preferred embodiment, a plant thus comprises QTL3 and Tm-1 homozygously and QTL2 heterozygously.

The tolerance QTLs according to the invention, namely QTL1, QTL2 and QTL3, conferring the resistance to TBRFV when combined with the Tm-1 gene, and conferring tolerance to TBRFV in the absence of such a combination, are chosen from the ones present in the genome of seeds of HAZTBRFVRES1. A sample of this S. lycopersicum seed has been deposited by Hazera Seeds Ltd. Berurim, M. P. Shikmim 79837, Israel, pursuant to and in satisfaction of the requirements of the Budapest treaty on the International Recognition of the deposit of Microorganisms for the Purpose of Patent procedure (“the Budapest Treaty” with the National collection of Industrial, Food and Marine bacteria (NCIMB) (NCIMB, Ltd, Ferguson Building, Craibstone Estate, Bucksburn, Aberdeen AB21 9YA, united Kingdom) on 16 May 2017 under accession number 42758. A deposit of this tomato seed is maintained by Hazera Seeds Ltd. Berurim, M. P. Shikmim 79837, Israel.

The tolerance QTLs conferring the tolerance to TBRFV, and conferring the resistance when combined with Tm-1 gene, are located on chromosome 6 for QTL1, on chromosome 9 for QTL2 and on chromosome 11 for QTL3. They are more preferably located within a chromosomal interval of chromosome 6 which comprises the SNP TO-0005197 (SEQ ID No:1) and the SNP TO-0145581 (SEQ ID No:2) for QTL1, within a chromosomal interval of chromosome 9 which comprises the SNP TO-0180955 (SEQ ID No:3) and the SNP TO-0196109 (SEQ ID No:6) for QTL2 and within a chromosomal interval of chromosome 11 which comprises the SNP TO-0122252 (SEQ ID No:7) and the SNP TO-0162427(SEQ ID No:18) for QTL3.

The specific polymorphisms corresponding to the SNPs (Single Nucleotide Polymorphism) referred to in this description, as well as the flanking sequences of these SNPs in the S. lycopersicum genome, are given in the experimental section (see tables 3 and 4) and the accompanying sequence listing. Their location with respect to the version 2.40 of the tomato genome, on chromosomes 6, 9 and 11, is indicated in table 3 and their flanking sequences are also illustrated in table 4, and in the sequence listing.

It is to be noted in this respect that, by definition, a SNP refers to a single nucleotide in the genome, which is variable depending on the allele which is present, whereas the flanking nucleotides are identical. For ease of clear identification of the position of the different SNPs, their position is given in tables 3 and 4, by reference to the tomato genome sequence in its version 2.40 and by reference to their flanking sequences, identified by SEQ ID number. In the sequence associated with a specific SNP in the present application, for example SEQ ID No:1 for the SNP TO-0005197, only one nucleotide within the sequence actually corresponds to the polymorphism, namely the 61^{st} nucleotide of SEQ ID No:1 corresponds to the polymorphic position of SNP TO-0005197, which can be T or C as indicated in table 4. The flanking sequences are given for positioning the SNP in the genome but are not part of the polymorphism as such.

The present inventors have identified that the tolerance QTLs responsible for the resistance or enhanced tolerance when combined with the Tm-1 gene, are to be found in the chromosomal regions mentioned above, by identifying the presence of sequences at different loci along said region, namely at 18 different loci defined by the 18 following SNPs: TO-0005197 (SEQ ID No:1) and TO-0145581 (SEQ ID No:2) for QTL1 on chromosome 6, TO-0180955 (SEQ ID No:3), TO-0196724 (SEQ ID No:4), TO-0145125 (SEQ ID No:5) and TO-0196109 (SEQ ID No:6) for QTL2 on chromosome 9 and TO-0122252 (SEQ ID No:7), TO-0144317 (SEQ ID No:8), TO-0142270 (SEQ ID No:9), TO-0142294 (SEQ ID No:10), TO-0142303 (SEQ ID No:11), TO-0142306 (SEQ ID No:12), TO-0182276 (SEQ ID No:13), TO-0181040 (SEQ ID No:14), TO-0123057 (SEQ ID No:15), TO-0125528 (SEQ ID No:16), TO-0162432 (SEQ ID No:17) and TO-0162427 (SEQ ID No:18) for QTL3 on chromosome 11.

These 18 SNPs are associated or genetically linked to at least one of the tolerance QTL. By association, or genetic association, and more specifically genetic linkage, it is to be understood that a genetic polymorphism of the marker (i.e. a specific allele of the SNP marker) and the phenotype of interest occur simultaneously, i.e. are inherited together, more often than would be expected by chance occurrence, i.e. there is a non-random association of the allele and of the genetic sequences responsible for the phenotype, as a result of their proximity on the same chromosome.

A molecular marker of the invention, either one of 18 markers disclosed above or alternative markers, are inherited with the phenotype of interest in preferably more than 90% of the meioses, preferably in more than 95%, 96%, 98% or 99% of the meioses.

According to another embodiment of the invention, the tolerance QTLs present in the genome of a plant, seed or cell of the invention are preferably to be found at least at one or more of the 18 loci encompassing said 18 SNPs mentioned above, namely the locus encompassing TO-0005197 (SEQ ID No:1), the locus encompassing TO-0145581 (SEQ ID No:2) for QTL1 on chromosome 6, the locus encompassing TO-0180955 (SEQ ID No:3), the locus encompassing TO-0196724 (SEQ ID No:4), the locus encompassing TO-0145125 (SEQ ID No:5), the locus encompassing TO-0196109 (SEQ ID No:6), for QTL2 on chromosome 9, the locus encompassing TO-0122252 (SEQ ID No:7), the locus encompassing TO-0144317 (SEQ ID No:8), the locus encompassing TO-0142270 (SEQ ID No:9), the locus encompassing TO-0142294 (SEQ ID No:10), the locus encompassing TO-0142303 (SEQ ID No:11), the locus encompassing TO-0142306 (SEQ ID No:12), the locus encompassing TO-0182276 (SEQ ID No:13), the locus encompassing TO-0181040 (SEQ ID No:14), the locus encompassing TO-0123057 (SEQ ID No:15), the locus encompassing TO-0125528 (SEQ ID No:16), the locus encompassing TO-0162432 (SEQ ID No:17) and the locus encompassing TO-0162427 (SEQ ID No:18) for QTL3 on chromosome 11.

In an embodiment, in a tomato plant according to the invention, the QTLs present in the genome of a plant, seed or cell of such tomato plant, and which are to be combined with the Tm-1 gene, are preferably to be found at least at one or more of the following loci: the locus encompassing TO-0005197, the locus encompassing TO-0145581 for QTL1 on chromosome 6, and/or the locus encompassing TO-0180955, the locus encompassing TO-0196724, the locus encompassing TO-0145125 and the locus encompassing TO-0196109 for QTL2 on chromosome 9.

In another embodiment of the invention, the QTLs present in the genome of a plant, seed or cell of the tomato plant, which are to be combined with the Tm-1 gene, are preferably to be found at least at one or more of the following loci: the locus encompassing TO-0122252, the locus encompassing TO-0144317, the locus encompassing TO-0142270, the locus encompassing TO-0142294, the locus encompassing TO-0142303, the locus encompassing TO-0142306, the locus encompassing TO-0182276, the locus encompassing TO-0181040, the locus encompassing TO-0123057, the locus encompassing TO-0125528, the locus encompassing TO-0162432 and the locus encompassing TO-0162427 for QTL3 on chromosome 11.

The alleles of the 18 SNPs linked to the tolerance QTLs conferring the TBRFV tolerance are allele T of TO-0005197, allele C of TO-0145581, allele G of TO-0180955, allele C of TO-0196724, allele G of TO-0145125, allele G of TO-0196109, allele T of TO-0122252, allele C of TO-0144317, allele T of TO-0142270, allele G of TO-0142294, allele A of TO-0142303, allele A of TO-0142306, allele G of TO-0182276, allele G of TO-0181040, allele G of TO-0123057, allele A of TO-0125528, allele C of TO-0162432 and allele T of TO-0162427. The presence of the tolerance QTLs can be revealed by the presence of said specific alleles. The alleles of these SNPs can thus reflect the presence of the tolerance QTLs according to the invention, which are to be combined with the Tm-1 gene.

According to a preferred embodiment of the present invention, the QTLs conferring the tolerance to TBRFV, which are to be combined with the Tm-1 gene, are on one or more chromosomal intervals delimited by the SNPs as disclosed. According to this embodiment, the QTL1 is on a chromosomal interval of chromosome 6 delimited on one side by SNP TO-0005197 and on the other side by SNP TO-0145581.

According to another embodiment, the QTL2 is on a chromosomal interval of chromosome 9 delimited on one side by SNP TO-0180955 and on the other side by SNP TO-0196109.

According to another embodiment, the QTL3 is on a chromosomal interval of chromosome 11 delimited on one side by SNP TO-0122252 and on the other side by TO-0162427. More preferred chromosomal intervals of chromosome 11 within which QTL3 is to be found are the interval delimited by TO-0144317 and TO-0125528, the interval delimited by TO-0142270 and TO-0162432, the interval delimited by TO-0144317 and TO-0162432, and the interval delimited by TO-0142270 and TO-0125528. The even more preferred interval is the interval delimited by TO-0142270 and TO-0125528. Another preferred interval is the interval delimited by and comprising TO-0142294 and TO-0125528.

It is noted in this respect that specific positions in a chromosome can indeed be defined with respect to single nucleotide polymorphism, insofar as the flanking sequences of said SNPs are defined in order to unambiguously position them on the genome. The present inventors have used SNPs, identified by their flanking sequences, with different alleles, to identify and follow the QTLs of the present invention.

A chromosomal region delimited by two SNPs X and Y refers to the section of the chromosome lying between the positions of these two SNPs and comprising said SNPs, therefore the nucleotide sequence of this chromosomal region begins with the nucleotide corresponding to SNP X and ends with the nucleotide corresponding to SNP Y, i.e. the SNPs are comprised within the region they delimit, in the sense of the invention.

In a plant, seed or cell of the invention, the presence of the tolerance QTLs, which are to be combined with the Tm-1 resistance gene, is preferably characterized by TO-0005197 and/or TO-0145581 for the QTL1 on chromosome 6 and/or by TO-0180955, TO-0196724, TO-0145125 and/or TO-0196109 for the QTL2 on chromosome 9 and/or by TO-0122252, TO-0144317, TO-0142270, TO-0142294, TO-0142303, TO-0142306, TO-0182276, TO-0181040, TO-0123057, TO-0125528, TO-0162432 and TO-0162427, most preferably by TO-0142294, TO-0142303, TO-0142306, TO-0182276, TO-0181040, TO-0123057, TO-0125528, and even more preferably TO-0182276, for the QTL3 on chromosome 11.

When present homozygously in the genome of a tomato plant, QTL1 and/or QTL2 will confer independently and collectively fruit tolerance to TBRFV and QTL3 will confer leaf tolerance to TBRFV, unless combined with the Tm-1 resistance gene in order to confer resistance or enhanced tolerance to TBRFV according to the invention.

The tolerance QTLs as defined above are in combination with the Tm-1 gene, in the genome of a plant, seed or cell of the invention.

The Tm-1 gene may be present heterozygously or homozygously in the genome of a plant, seed or cell of the invention. It is however preferred that said gene be present homozygously. The present inventors have also found suitable markers for detecting the presence of the Tm-1 gene in the genome of a plant, seed or cell of the invention. The presence of the Tm-1 resistance gene, which is to be combined with one or more tolerance QTLs, is preferably characterized by the SNP TO-0200838 (SEQ ID No: 21).

The allele of the SNP TO-0200838 corresponding to the Tm-1 gene is allele A of TO-0200838. The presence of the Tm-1 gene conferring the resistance to TBRFV when combined with at least one tolerance QTL can be revealed by the presence of said specific allele.

According to an embodiment, a S. lycopersicum plant, cell or seed according to the invention also comprises in its genome a Tm-2 resistance gene, especially Tm-2 or Tm-2² (also known as Tm-2a) allele. The Tm-2 and Tm-2² alleles are well-known to the skilled reader and described in detail in the literature. Such a Tm-2 or Tm-2² allele is either found homozygously or heterozygously in the genome of a plant, cell or seed according to the invention, but preferably heterozygously.

In a preferred embodiment, a plant of the invention comprises a Tm-2 gene, preferably a Tm-2² allele, on one of chromosome 9 homolog and QTL2 on the other homolog. Such a plant moreover comprises homozygously at least one of QTL1 and QTL3, more preferably QTL3. This plant also comprises the Tm-1 gene, either homozygously or heterozygously. Alternatively, although less preferred, a plant, seed or cell according to the invention does not exhibit TMV or ToMV resistance insofar as the Tm-1 resistance gene does not provide TMV or ToMV resistance to most of the circulating TMV and ToMV strains, especially it does not comprise a Tm-2 resistance gene.

The invention encompasses tomato plants, comprising for example the genotype combinations according to table 1, wherein “Horn” means homozygous for the tolerance QTL or resistance gene, “Het” heterozygous for the tolerance QTL or resistance gene and “0” absence of the tolerance QTL or resistance gene.

TABLE 1
preferred genotypes according to the invention:

| # | QTL1 | QTL2 | QTL3 | Tm-1 |
|---|---|---|---|---|
| 1 | Hom | ∅ | ∅ | Het |
| 2 | ∅ | Hom | ∅ | Het |
| 3 | ∅ | ∅ | Hom | Het |
| 4 | Hom | ∅ | ∅ | Hom |
| 5 | ∅ | Hom | ∅ | Hom |
| 6 | ∅ | ∅ | Hom | Hom |
| 7 | Het | ∅ | Hom | Het |
| 8 | ∅ | Het | Hom | Het |
| 9 | Het | Het | Hom | Het |
| 10 | Het | ∅ | Hom | Hom |
| 11 | ∅ | Het | Hom | Hom |
| 12 | Het | Het | Hom | Hom |
| 13 | Hom | ∅ | Hom | Het |
| 14 | ∅ | Hom | Hom | Het |
| 15 | Hom | ∅ | Hom | Hom |
| 16 | ∅ | Hom | Hom | Hom |
| 17 | Hom | Het | Hom | Het |
| 18 | Hom | Het | Hom | Hom |
| 19 | Hom | Het | ∅ | Het |
| 20 | Hom | ∅ | Het | Het |
| 21 | Hom | Het | Het | Het |
| 22 | Hom | Het | ∅ | Hom |
| 23 | Hom | ∅ | Het | Hom |
| 24 | Hom | Het | Het | Hom |
| 25 | Het | Het | Het | Hom |
| 26 | Het | Het | Het | Het |

The presence at the homozygous or heterozygous state of the tolerance QTLs and Tm-1 gene can be detected with the different SNP markers disclosed in the present description.

Preferably, a S. lycopersicum plant according to the invention is a commercial plant or line. Such a commercial plant or line preferably also exhibits additional resistances such as nematode resistance trait (Mi-1 or Mi-j), as well as Fusarium and Verticillium resistances.

Other resistances or tolerances are also envisaged according to the invention.

According to a preferred embodiment, a plant of the invention is not resistant to Pepino Mosaic Virus (PepMV). According to another embodiment, a tomato plant of the invention is also resistant to PepMV.

According to still another embodiment, a plant of the invention is a determinate, indeterminate or semi-indeterminate plant, or seed or cell thereof, i.e. corresponding to determinate, indeterminate or semi-indeterminate growth habit.

By determinate, it is meant tomato plants which tend to grow their foliage first, then set flowers that mature into fruit if pollination is successful. All of the fruits tend to ripen on a plant at about the same time. Indeterminate tomatoes start out by growing some foliage, then continue to produce foliage and flowers throughout the growing season. These plants will tend to have tomato fruit in different stages of maturity at any given time. The semi-determinate tomatoes have a phenotype between determinate and indeterminate, they are typical determinate types except that grow larger than determinate varieties.

According to still another embodiment, a plant of the invention is used as a scion or as a rootstock in a grafting process. Grafting is a process that has been used for many years in crops such as cucurbitacea, but only more recently for tomato. Grafting may be used to provide a certain level of resistance to telluric pathogens or to certain nematodes. Grafting is therefore intended to prevent contact between the plant or variety to be cultivated and the infested soil. The variety of interest used as the graft or scion, optionally an F1 hybrid, is grafted onto the resistant plant used as the rootstock. The resistant rootstock remains healthy and provides, from the soils, the normal supply for the graft that it isolates from the diseases.

Moreover, the commercial plant of the invention gives rise to fruits in suitable conditions, which are at least 25 grams at full maturity, preferably at least 100 g at full maturity and or even more preferred at least 200 g at full maturity.

As detailed above, the invention is directed to S. lycopersicum plants, exhibiting the TBRFV resistance phenotype, as well as to seeds giving rise to those plants.

A plant or seed according to the invention may be a progeny or offspring of an hybrid between a plant grown from the deposited seeds HAZTBRFVRES1, deposited at the NCIMB under the accession number NCIMB 42758 and a S. lycopersicum plant bearing the Tm-1 gene. Plants grown from the deposited seeds are indeed homozygous for the tolerance QTLs, they thus bear in their genome the QTLs of interest on each of the homologues of chromosome 6, 9 and 11. They can be used to combine these QTLs with the Tm-1 gene, as illustrated in the examples of the present application, by crossing, selfing and/or backcrossing steps.

With regard to the deposited seeds of HAZTBRFVRES1 (NCIMB 42758), it is noted that these seeds do not correspond to a plant variety, they are not homozygous for most of the genes except the tolerance QTLs; their phenotype is thus not fixed during propagation, except for the foliar and fruit tolerance of the invention; such that their phenotypic traits segregate during propagation, with the exception of TBRFV foliar and fruit tolerance.

According to an embodiment of the invention, the plant, seed or cell is resistant more specifically to the Israeli strain of TBRFV. By Israeli strain of TBRFV, it is meant a strain of TBRFV as first identified and sequenced by Luria et al, namely a strain infecting tomatoes and having a sequence with a very high degree of sequence identity with KX619418 (SEQ ID No:26), namely a degree of sequence identity with SEQ ID No:26 which is higher than the degree of sequence identity with SEQ ID No:25, thus a sequence identity above 99%, preferably above 99.5% or even above. A plant, seed or cell of the invention is thus according to an embodiment more resistant to the Israeli strain than to the Jordanian strain, for example it is resistant only to the Israeli strain.

The invention is also directed to plants or seeds obtainable by transferring the tolerance QTLs from a S. lycopersicum plant comprising the tolerance QTLs, representative seeds thereof were deposited under NCIMB accession NCIMB-42758, into another S. lycopersicum genetic background comprising the Tm-1 gene, for example by crossing said plant with a tomato plant parent comprising the Tm-1 gene, and selection of plants bearing the tolerance QTLs, or at least one of them, and the Tm-1 gene. In such crossing, QTL1, QTL2 and/or QTL 3 or any combination thereof could be transferred. According to one embodiment, QTL1 only, or QTL2 only, or both QTLs 1 and 2 are transferred. According to another embodiment, QTL 3 is transferred. Alternatively, QTL1 and QTL3, QTL2 and QTL3 or QTL1, QTL2 and QTL3, preferably QTL2 and QTL3 are transferred from the deposited seeds of HAZTBRFVRES1 (NCIMB 42758) into a tomato genetic background comprising the Tm-1 gene. Preferably, the obtained progeny is selfed, such that at least one tolerance QTL is homozygously present in the obtained genome.

According to a preferred embodiment, the plant comprises at least two QTLs chosen from QTL1,

QTL2 and QTL3, at least one being heterozygous; preferably at least one is homozygous. It is noted that the seeds or plants of the invention may be obtained by different processes, and are not exclusively obtained by means of an essentially biological process.

According to such an aspect, the invention relates to a tomato plant or seed, preferably a non-naturally occurring tomato plant or seed, which may comprise two or more mutations in its genome, which provides the plant with a Tomato Brown Rugose Fruit virus resistance, wherein at least one mutation is, for example, as present in the genome of plants of which a representative sample was deposited with the NCIMB under deposit number NCIMB 42758, and at least another mutation is on chromosome 2 and corresponds to the sequence of a Tm-1 gene.

In another embodiment, the invention relates to a method for obtaining a tomato plant or seed carrying two or more mutations in its genome, which provides the plant with a resistance to Tomato Brown Rugose Fruit virus. Such a method is illustrated in example 4 and may comprise:

a) treating M0 seeds of a tomato plant to be modified with a mutagenic agent to obtain M1 seeds;

b) growing plants from the thus obtained M1 seeds to obtain M1 plants;

c) producing M2 seeds by self-fertilisation of M1 plants; and

d) optionally repeating step b) and c) n times to obtain M1+n seeds.

The M1+n seeds are grown into plants and submitted to Tomato Brown Rugose Fruit virus infection. The surviving plants, or those with the milder symptoms of TBRFV infection, are multiplied one or more further generations while continuing to be selected for their resistance to Tomato Brown Rugose Fruit virus. The M0 seeds are for example from a tomato plant bearing the Tm-1 gene.

In this method, the M1 seeds of step a) can be obtained via chemical mutagenesis such as EMS mutagenesis. Other chemical mutagenic agents include but are not limited to, diethyl sufate (des), ethyleneimine (ei), propane sultone, N-methyl-N-nitrosourethane (mnu), N-nitroso-N-methylurea (NMU), N-ethyl-N-nitrosourea(enu), and sodium azide.

Alternatively, the mutations are induced by means of irradiation, which is for example selected from x-rays, fast neutrons, UV radiation.

In another embodiment of the invention, the mutations are induced by means of genetic engineering. Such mutations also include the integration of sequences corresponding to the tolerance QTLs and the Tm-1 gene, as well as the substitution of residing sequences by alternative sequences conferring the TBRFV resistance. Preferably, the mutations are the integration of one or more of QTL1, QTL2 and QTL3 as described above, in replacement of the homologous sequences of a S. lycopersicum plants, and integration of the Tm-1 gene, preferably on chromosome 2. Even more preferably, the mutation includes the substitution of the sequence comprised within SNP TO-0122252 (SEQ ID No:7) and SNP TO-0162427(SEQ ID No:18) on chromosome 11 of S. lycopersicum genome, or a fragment thereof, by the homologous sequence on chromosome 11 present in the genome of a plant of which a representative sample was deposited with the NCIMB under deposit number NCIMB 42758, and also includes the incorporation of the Tm-1 resistance gene, wherein the combination of the integrated sequences confer resistance to TBRFV. The substitution on chromosome 11 corresponding to QTL3 is preferably homozygous, the incorporation of Tm-1 gene can be homozygous or heterozygous.

The genetic engineering means which can be used include the use of all such techniques called New Breeding Techniques which are various new technologies developed and/or used to create new characteristics in plants through genetic variation, the aim being targeted mutagenesis, targeted introduction of new genes or gene silencing (RdDM). Example of such new breeding techniques are targeted sequence changes facilitated through the use of Zinc finger nuclease (ZFN) technology (ZFN-1, ZFN-2 and ZFN-3, see U.S. Pat. No. 9,145,565), Oligonucleotide directed mutagenesis (ODM), Cisgenesis and intragenesis, Grafting (on GM rootstock), Reverse breeding, Agro-infiltration (agro-infiltration “sensu stricto”, agro-inoculation, floral dip), Transcription Activator-Like Effector Nucleases (TALENs, see U.S. Pat. Nos. 8,586,363 and 9,181,535), the CRISPR/Cas system (see U.S. Pat. Nos. 8,697,359; 8,771,945; 8,795,965; 8,865,406; 8,871,445; 8,889,356; 8,895,308; 8,906,616; 8,932,814; 8,945,839; 8,993,233; and 8,999,641), engineered meganuclease re-engineered homing endonucleases, DNA guided genome editing (Gao et al., Nature Biotechnology (2016)), and Synthetic genomics. A major part of targeted genome editing, another designation for New Breeding Techniques, is the applications to induce a DNA double strand break (DSB) at a selected location in the genome where the modification is intended. Directed repair of the DSB allows for targeted genome editing. Such applications can be utilized to generate mutations (e.g., targeted mutations or precise native gene editing) as well as precise insertion of genes (e.g., cisgenes, intragenes, or transgenes). The applications leading to mutations are often identified as site-directed nuclease (SDN) technology, such as SDN1, SDN2 and SDN3. For SDN1, the outcome is a targeted, non-specific genetic deletion mutation: the position of the DNA DSB is precisely selected, but the DNA repair by the host cell is random and results in small nucleotide deletions, additions or substitutions. For SDN2, a SDN is used to generate a targeted DSB and a DNA repair template (a short DNA sequence identical to the targeted DSB DNA sequence except for one or a few nucleotide changes) is used to repair the DSB: this results in a targeted and predetermined point mutation in the desired gene of interest. As to the SDN3, the SDN is used along with a DNA repair template that contains new DNA sequence (e.g. gene). The outcome of the technology would be the integration of that DNA sequence into the plant genome. The most likely application illustrating the use of SDN3 would be the insertion of cisgenic, intragenic, or transgenic expression cassettes at a selected genome location. A complete description of each of these techniques can be found in the report made by the Joint Research Center (JRC) Institute for Prospective Technological Studies of the European Commission in 2011 and titled “New plant breeding techniques—State-of-the-art and prospects for commercial development”.

The invention in another aspect also concerns any plant likely to be obtained from seed or plants of the invention as described above, and also plant parts of such a plant, and most preferably explant, scion, cutting, seed, fruit, root, rootstock, pollen, ovule, embryo, protoplast, leaf, anther, stem, petiole, and any other plants part, wherein said plant, explant, scion, cutting, seed, fruit, root, rootstock, pollen, ovule, embryo, protoplast, leaf, anther, stem, petiole, and/or plant part is obtainable from a seed or plant according to the first aspect of the invention, i.e. bearing one, two or three of the tolerance QTLs of interest, in combination with the Tm-1 gene. These plant parts, inter alia explant, scion, cutting, seed, fruit, root, rootstock, pollen, ovule, embryo, protoplast, leaf, anther, stem or petiole, comprise in their genome the tolerance QTLs conferring the phenotype of fruit and/or foliar tolerance to TBRFV when present homozygously in the absence of the Tm-1 gene, and which confer TBRFV resistance when present in combination with the Tm-1 gene.

The tolerance QTLs referred to in this aspect of the invention are those defined above in the context of plants of the invention. The different features of the tolerance QTLs defined in relation with the first aspect of the invention apply mutatis mutandis to this aspect of the invention. The tolerance QTLs are thus preferably chosen from those present in the genome of a plant corresponding to the deposited material HAZTBRFVRES1 (NCIMB accession number 42758). They are advantageously characterized by the presence of allele T of TO-0005197, allele C of TO-0145581, allele G of TO-0180955, allele C of TO-0196724, allele G of TO-0145125, allele G of TO-0196109, allele T of TO-0122252, allele C of TO-0144317, allele T of TO-0142270, allele G of TO-0142294, allele A of TO-0142303, allele A of TO-0142306, allele G of TO-0182276, allele G of TO-0181040, allele G of TO-0123057, allele A of TO-0125528, allele C of TO-0162432 and/or allele T of TO-0162427, depending of the QTL of interest, and preferably by the presence of this or these alleles homozygously.

The presence of the Tm-1 resistance gene, which is to be combined with one or more tolerance QTLs, is preferably characterized by the SNP TO-0200838 (SEQ ID 21), more specifically by allele A of TO-0200838.

The TBRFV resistance is advantageously a resistance to the Israeli strain of TBRFV.

The invention is also directed to cells of S. lycopersicum plants, such that these cells comprise, in their genome, the combination of the Tm-1 gene, either homozygously or heterozygously, and at least one of the tolerance QTLs conferring the phenotype of fruit and/or foliar tolerance to TBRFV when present homozygously in the absence of the Tm-1 gene, and which confer TBRFV resistance when present in combination with the Tm-1 gene. The tolerance QTLs are those already defined in the description, they are characterized by the same features and preferred embodiments already disclosed with respect to the plants and seeds according to the preceding aspects of the invention. The presence of these tolerance QTLs can be revealed by the techniques disclosed above and well known to the skilled reader. It can inter alia be determined whether the QTLs are present homozygously or heterozygously in the genome of such a cell of the invention. They are advantageously characterized by the presence of allele T of TO-0005197, allele C of TO-0145581, allele G of TO-0180955, allele C of TO-0196724, allele G of TO-0145125, allele G of TO-0196109, allele T of TO-0122252, allele C of TO-0144317, allele T of TO-0142270, allele G of TO-0142294, allele A of TO-0142303, allele A of TO-0142306, allele G of TO-0182276, allele G of TO-0181040, allele G of TO-0123057, allele A of TO-0125528, allele C of TO-0162432 and/or allele T of TO-0162427, depending of the tolerance QTL of interest, and preferably by the presence of this or these alleles simultaneously on each chromosome, i.e. homozygously. Preferably at least one QTL is present homozygously and at least one QTL is present heterozygously.

According to an embodiment, the QTL2 on chromosome 9 is present heterozygously in a cell according to the invention. The other homolog of chromosome 9 according to a specific embodiment comprises a Tm-2 or Tm-2² gene or allele. Such a cell thus exhibits resistance to TMV/ToMV. Preferred genotypes for a cell of the invention are disclosed in table 1.

The presence of the Tm-1 resistance gene, which is to be combined with one or more tolerance QTLs, is preferably characterized by the SNP TO-0200838, more specifically by allele A of TO-0200838.

Cells according to the invention can be any type of S. lycopersicum cell, inter alia an isolated cell and/or a cell capable of regenerating a whole S. lycopersicum plant, bearing one or more of the tolerance QTLs of interest, preferably two tolerance QTLs, and the Tm-1 gene.

The present invention is also directed to a tissue culture of non-regenerable or regenerable cells of the plant as defined above according to the present invention; preferably, the regenerable cells are derived from embryos, protoplasts, meristematic cells, callus, pollen, leaves, anthers, stems, petioles, roots, root tips, fruits, seeds, flowers, cotyledons, and/or hypocotyls of the invention, and the cells contain the combination of the Tm-1 gene and one, two or three of the tolerance QTLs of interest, in whenever combination, homozygously or heterozygously in their genome, said QTLs conferring, when present homozygously fruit tolerance to TBRFV for QTL1 and/or QTL2, foliar tolerance to TBRFV for QTL3, and confer when present in combination with Tm-1, resistance or enhanced tolerance to TBRFV.

The tissue culture will preferably be capable of regenerating plants having the physiological and morphological characteristics of the foregoing tomato plant, and of regenerating plants having substantially the same genotype as the foregoing tomato plant. The present invention also provides tomato plants regenerated from the tissue cultures of the invention.

The invention also provides a protoplast of the plant defined above, or from the tissue culture defined above, said protoplast containing the combination of the Tm-1 gene and the tolerance QTLs conferring the TBRFV resistance.

According to another aspect, the present invention is also directed to the use of a tomato plant of the invention, comprising preferably homozygously at least one of the QTLs of the invention and also comprising the Tm-1 gene, also preferably homozygously, as a breeding partner in a breeding program for obtaining S. lycopersicum plants having TBRFV resistance. Indeed, such a breeding partner harbors homozygously in its genome at least one of the tolerance QTLs. By crossing this plant with a tomato plant, especially a line, it is thus possible to transfer one, two or the three tolerance QTLs, as well as the Tm-1 gene, to the progeny. A plant according to the invention can thus be used as a breeding partner for introgressing the tolerance QTLs and the Tm-1 gene into a S. lycopersicum plant or germplasm. Although a plant or seed bearing the tolerance QTLs or the Tm-1 gene heterozygously, can also be used as a breeding partner as detailed above, the segregation of the phenotype is likely to render the breeding program more complex.

The introgressed tolerance QTLs and Tm-1 gene will advantageously be introduced into varieties that contain other desirable genetic traits such as resistance to disease, especially resistance to TMV/ToMV, early fruit maturation, drought tolerance, fruit shape, and the like.

The invention is also directed to the use of a plant or seed comprising at least one of the tolerance QTLs, preferably homozygously as for example a plant or seed of S. lycopersicum, deposited at the NCIMB under the accession number NCIMB 42758, or progeny thereof, bearing homozygously the QTLs conferring the tolerance to TBRFV infection, as a breeding partner in a breeding program with S. lycopersicum plants comprising the Tm-1 gene. Such a breeding program allows to obtain S. lycopersicum plant or seed resistant to TBRFV.

In such a breeding program, the selection of the progeny displaying the desired phenotype of TBRFV resistance, or bearing at least one of the tolerance QTLs and the Tm-1 gene, can advantageously be carried out on the basis of the alleles of the SNP markers, especially the SNP markers disclosed above.

A progeny of the plant is preferably selected on the presence of allele T of TO-0005197 and/or allele C of TO-0145581 for the presence of QTL1 on chromosome 6, allele G of TO-0180955, allele C of TO-0196724, allele G of TO-0145125 and/or allele G of TO-0196109 for the presence of QTL2 on chromosome 9, allele T of TO-0122252, allele C of TO-0144317, allele T of TO-0142270, allele G of TO-0142294, allele A of TO-0142303, allele A of TO-0142306, allele G of TO-0182276, allele G of TO-0181040, allele G of TO-0123057, allele A of TO-0125528, allele C of TO-0162432 and/or allele T of TO-0162427 for the presence of QTL3 on chromosome 11. The progeny of the plant is preferably selected on the presence of the same allele on both homologues of each chromosome.

With respect to the presence of the Tm-1 gene, a progeny is preferably selected on the basis of allele A of TO-0200838.

The selection can alternatively be made on the basis of the presence of any one of the alleles of the 18 SNPs linked to tolerance QTLs, or a combination of these alleles, in addition to the selection on the presence of the Tm-1 gene. Such selection will be made on the presence of the alleles of interest in a genetic material sample of the plant to be selected. The presence of these alleles indeed confirms the presence of the tolerance QTLs at the loci defined by said SNPs. Moreover, further to point mutation or recombination event, it is conceivable that at least 1 or 2 of these alleles is lost, the remaining of the chromosomal fragment bearing the tolerance QTLs.

A plant according to the invention, is thus particularly valuable in a marker assisted selection for obtaining commercial tomato lines and varieties, having the improved phenotype of the invention.

The invention is also directed to a method for identifying, detecting and/or selecting S. lycopersicum plants resistant to TBRFV, capable of inhibiting, reducing or delaying the replication of the virus and/or reducing the virus titer in the plant. Such a method comprises the step of detecting, in a plant to be tested or selected, the combination of the Tm-1 gene and at least one of the tolerance QTLs, wherein said QTL(s) is preferably present homozygously. The method may thus comprise the detection of at least one of the following markers: allele T of TO-0005197, allele C of TO-0145581, allele G of TO-0180955, allele C of TO-0196724, allele G of TO-0145125, allele G of TO-0196109, allele T of TO-0122252, allele C of TO-0144317, allele T of TO-0142270, allele G of TO-0142294, allele A of TO-0142303, allele A of TO-0142306, allele G of TO-0182276, allele G of TO-0181040, allele G of TO-0123057, allele A of TO-0125528, allele C of TO-0162432 or allele T of TO-0162427, preferably in homozygous state, in a genetic material sample of the plant to be identified and/or selected, as well as the detection of the Tm-1 gene. Preferably, the tolerance QTL is QTL3 and it is detected by the presence of T of TO-0122252, allele C of TO-0144317, allele T of TO-0142270, allele G of TO-0142294, allele A of TO-0142303, allele A of TO-0142306, allele G of TO-0182276, allele G of TO-0181040, allele G of TO-0123057, allele A of TO-0125528, allele C of TO-0162432 and allele T of TO-0162427, preferably in homozygous state.

Advantageously, the method comprises the detection of two tolerance QTLs, at least one being heterozygous, and preferably at least one being homozygous. Preferably, QTL2 is present heterozygously and QTL3 is present homozygously.

The invention is also directed to a method for detecting or selecting S. lycopersicum plants having at least one of the tolerance QTL in combination with the Tm-1 gene, i.e. having the marker alleles disclosed in this description, wherein the detection or selection is made on condition of TBRFV infection comprising inoculation of TBRFV on the plants to be tested, and detection of the inhibition, reduction or delay of the viral replication and/or reduction of the virus titer in the plant.

The invention is further directed to a method for detecting and or selecting S. lycopersicum plants having the Tm-1 gene and at least one of the tolerance QTLs, wherein the detection of the tolerance QTL is based on the detection of any molecular marker revealing the presence of said QTLs. Indeed, the identification and then the use of molecular markers, distinct from the 18 SNPs disclosed above, can be easily done by the skilled artisan. The tolerance QTLs can thus be identified through the use of different, alternative markers.

The invention is thus also directed to a method for detecting and/or selecting S. lycopersicum plants resistant to TBRFV, inhibiting, reducing or delaying the replication of the virus, said method comprising the steps of:
a) Assaying tomato plant for the combination in its genome of
   the presence of the Tm-1 resistance gene on chromosome 2, and
   the presence of at least one genetic marker genetically linked to a tolerance QTL chosen from QTL3 on chromosome 11, QTL1 on chromosome 6 and QTL2 on chromosome 9,
b) Selecting a plant comprising in its genome the Tm-1 gene and the genetic marker, and the tolerance QTL linked to said genetic marker,
wherein the chosen QTL and the genetic marker are to be found, for QTL1, on chromosome 6, within the chromosomal region delimited by TO-0005197 (SEQ ID No:1) and TO-015581 (SEQ ID No:2), for QTL2, on chromosome 9, within the chromosomal region delimited by TO-0180955 (SEQ ID No:3) and TO-0196109 (SEQ ID No:6) and for QTL3, on chromosome 11, within the chromosomal region delimited by TO-0122252 (SEQ ID No:7) and TO-0162427 (SEQ ID No:18).

The genetic marker under consideration is preferably a SNP marker. The tolerance QTLs are as defined in this description, and as found in the genome of a plant of the seeds HAZTBRFVRES1 NCIMB accession number 42758.

According to a still another aspect, the invention also concerns methods or processes for the production of S. lycopersicum plants having TBRFV resistance, especially commercial plants and inbred parental lines. The present invention is indeed also directed to transferring one or more of the tolerance QTLs and/or the Tm-1 gene, in order to confer TBRFV resistance, to other tomato varieties, or other species or inbred parental lines, especially resistance to the Israeli strain of TBRFV, and is useful for producing new types and varieties of tomatoes. These methods comprise the transfer of at least one tolerance QTL and Tm-1 gene to another plant, as well as transfer of at least one tolerance QTL to another Tm-1 bearing plant.

A method or process for the production of a plant having these features may for example comprise the following steps:

a) Crossing a plant grown from a deposited seeds NCIMB 42758, or progeny thereof, bearing QTL1, QTL2 and/or QTL3 conferring TBRFV tolerance, and a S. lycopersicum plant, preferably devoid of said QTL(s), and bearing the Tm-1 gene,

b) Selecting a plant in the progeny thus obtained, bearing one, two or three of the tolerance QTL1, QTL2 and/or QTL3 in combination with the Tm-1 gene;

c) Optionally self-pollinating one or several times the plant obtained at step b) and selecting in the progeny thus obtained a plant having resistance to TBRFV.

The TBRFV resistance delays, reduces or inhibits the replication or multiplication of the TBRF virus, and/or reduces the virus titer in the plant.

Alternatively, the method or process may comprise instead of step a) the following steps:
a1) Crossing a plant grown from the deposited seeds NCIMB 42758 or progeny thereof, bearing QTL1, QTL2 and/or QTL3 conferring TBRFV tolerance, and a S. lycopersicum plant, preferably devoid of said QTL(s), and bearing the Tm-1 gene, thus generating F1 hybrids,
a2) Increasing the F1 hybrid by means of selfing to create F2 population,

In the above methods or processes, SNPs markers are preferably used in steps b) and/or c), for selecting plants bearing the tolerance QTL and/or the Tm-1 gene.

The SNP markers for the tolerance QTLs are preferably one or more of the 18 SNP markers already disclosed in the present description, including all combinations thereof as mentioned elsewhere in the application.

According to a preferred embodiment, the selection for plants having a tolerance QTL is made on the basis of TO-0182276, or on the basis of at least one of TO-0142294, TO-0142303, TO-0142306, TO-0182276, TO-0181040, TO-0123057, TO-0125528.

By selecting a plant on the basis of the allele of one or more SNPs, it is to be understood that the plant is selected as having a tolerance QTL when the allele of the SNP(s) is (are) the allele corresponding to the allele of the HAZTBRFVRES1 parent for this SNP and not the allele of the initial S. lycopersicum plant devoid of said QTLs. For example, a plant can be selected as having the tolerance QTLs of the invention, when allele T of TO-0005197, allele C of TO-0145581, allele G of TO-0180955, allele C of TO-0196724, allele G of TO-0145125, allele G of TO-0196109, allele T of TO-0122252, allele C of TO-0144317, allele T of TO-0142270, allele G of TO-0142294, allele A of TO-0142303, allele A of TO-0142306, allele G of TO-0182276, allele G of TO-0181040, allele G of TO-0123057, allele A of TO-0125528, allele C of TO-0162432 and/or allele T of TO-0162427 is detected.

Preferably, the S. lycopersicum plant of step a) or a1) is an elite line, used in order to obtain a plant with commercially desired traits or desired horticultural traits. This plant has preferably been previously modified in order to incorporate the Tm-1 gene. According to an embodiment, this plant is susceptible to TBRFV. This plant preferably comprises the Tm-1 gene, and preferably also the Tm-2 gene, or its Tm-2² allele.

The selection of plants bearing the Tm-1 gene is preferably carried out by detection of allele A of SNP TO-0200838.

A method or process as defined above may advantageously comprise backcrossing steps, preferably after step c), in order to obtain plants having all the characterizing features of S. lycopersicum plants. Consequently, a method or process for the production of a plant having these features may also comprise the following additional steps:

d) Backcrossing the resistant plant selected in step b) or c) with a S. lycopersicum plant;

e) Selecting a plant bearing one, two or three of the tolerance QTL1, QTL2 and/or QTL3 in combination with the Tm-1 gene.

The plant used in step a), namely the plant corresponding to the deposited seeds can be a plant grown from the deposited seeds; it may alternatively be any plant according to the 1^{st} aspect of the invention, bearing the QTLs conferring the phenotype, preferably bearing at least one of these sequences homozygously. In such a case, the plant is crossed in step a) with a S. lycopersicum plant, preferably devoid of said QTL(s), but not necessarily bearing the Tm-1 gene.

At step e), SNPs markers can be used for the selection of plants bearing a tolerance QTL and a Tm-gene; the SNP markers which can be used are for example those described in the previous sections of this description.

It is to be noted that, when plants having homozygously at least one tolerance QTL are to be selected, the selection is to be made on the basis of one or more of the SNPs linked to the tolerance QTLs, on the presence of the alleles representative of the QTLs, namely the alleles of the HAZTBRFVRES1 parent, coupled to the absence of the alleles representative of the recurrent susceptible S. lycopersicum parent. When plants having heterozygously at least one tolerance QTL are to be selected, the selection is to be made on the basis of one or more of the SNPs linked to the tolerance QTLs, on the presence of both alleles of the SNPs, i.e. the allele of the HAZTBRFVRES1 parent, and the allele of the recurrent susceptible S. lycopersicum parent.

The plant selected at step b), c) or e) is preferably a commercial plant, especially a plant having fruits which weigh at least 25 g, at least 100 g or at least 200 g at full maturity in normal culture conditions. Preferably, steps d) and e) are repeated at least twice and preferably three times, not necessarily with the same S. lycopersicum plant. Said S. lycopersicum plant is preferably a breeding line. Resistance to nematode trait or resistance to ToMV may additionally be selected, at each selection step of the processes disclosed above.

The self-pollination and backcrossing steps may be carried out in any order and can be intercalated, for example a backcross can be carried out before and after one or several self-pollinations, and self-pollinations can be envisaged before and after one or several backcrosses.

The selection of the progeny having the desired TBRFV resistance, which delays, reduces and/or inhibits the replication of the virus, and/or reduces the virus titer in the plant, can also be made on the basis of the comparison of the Tomato Brown Rugose Fruit virus resistance from the S. lycopersicum parent, through protocols as disclosed inter alia in the examples.

The method used for allele detection can be based on any technique allowing the distinction between two different alleles of a SNP, on a specific chromosome.

The present invention also concerns a plant obtained or obtainable by such a method. Such a plant is indeed a S. lycopersicum plant having the TBRFV resistance according to the first aspect of the invention.

In all the methods and processes according to the invention, the initial TBRFV-susceptible S. lycopersicum plant can be determinate, indeterminate or semi-determinate.

As already disclosed, the tomato plants according to the invention are preferably also resistant to Tomato Mosaic Virus, to nematodes, and to Fusarium and Verticillium. In order to obtain such plants in the processes and methods of the invention, the S. lycopersicum parents used in the breeding schemes are preferably bearing sequences conferring resistance to Tomato Mosaic Virus, to nematodes, and to Fusarium and Verticillium; and the selection steps are carried out to select plants having these resistance sequences, in addition to the tolerance QTL(s) and Tm-1 gene.

The invention is also directed to a method for breeding S. lycopersicum plants having resistance to TBRFV, comprising the steps of crossing a plant grown from the deposited seeds NCIMB 42758 or progeny thereof bearing QTL1, QTL2 and/or QTL3 conferring TBRFV tolerance, with a S. lycopersicum plant bearing the Tm-1 gene.

The present invention is also directed to a S. lycopersicum plant and seed obtainable by any of the methods and processes disclosed above.

Any S. lycopersicum seed of the invention is preferably coated or pelleted with individual or combined active species such as plant nutrients, enhancing microorganisms, or products for disinfecting the environment of the seeds and plants. Such species and chemicals may be a product that promotes the growth of plants, for example hormones, or that increases their resistance to environmental stresses, for example defense stimulators, or that stabilizes the pH of the substrate and its immediate surroundings, or alternatively a nutrient.

They may also be a product for protecting against agents that are unfavorable toward the growth of young plants, including herein viruses and pathogenic microorganisms, for example a fungicidal, bactericidal, hematicidal, insecticidal or herbicidal product, which acts by contact, ingestion or gaseous diffusion; it is, for example, any suitable essential oil, for example extract of thyme. All these products reinforce the resistance reactions of the plant, and/or disinfect or regulate the environment of said plant. They may also be a live biological material, for example a nonpathogenic microorganism, for example at least one fungus, or a bacterium, or a virus, if necessary with a medium ensuring its viability; and this microorganism, for example of the Pseudomonas, Bacillus, Trichoderma, Clonostachys, Fusarium, Rhizoctonia, etc. type stimulates the growth of the plant, or protects it against pathogens.

In all the previous methods and processes, the identification of the plants bearing homozygously the tolerance QTLs could be done by the detection of at least one of the alleles linked with each of the QTLs, but also in combination with the absence of the other allelic form of the SNPs of the present invention. As such, the identification of a plant bearing homozygously QTL3 of the present invention will be based on the identification of allele T of TO-0122252, and/or allele C of TO-0144317, and/or allele T of TO-0142270, and/or allele G of TO-0142294, and/or allele A of TO-0142303, and/or allele A of TO-0142306, and/or allele G of TO-0182276, and/or allele G of TO-0181040, and/or allele G of TO-0123057, and/or allele A of TO-0125528, and/or allele C of TO-0162432 and/or allele T of TO-0162427 as well as the absence of allele A of TO-0122252, allele T of TO-0144317, allele C of TO-0142270, allele A of TO-0142294, allele C of TO-0142303, allele G of TO-0142306, allele A of TO-0182276, allele A of TO-0181040, allele T of TO-0123057, allele G of TO-0125528, allele T of TO-0162432 and allele C of TO-0162427.

When plants bearing heterozygously one of the tolerance QTL, preferably bearing heterozygously the tolerance QTL2 are to be selected, the identification implies the detection of allele G of TO-0180955 and/or allele C of TO-0196724 and/or allele G of TO-0145125 and/or allele G of TO-0196109 as well as, simultaneously the detection of allele A of TO-0180955, allele T of TO-0196724, allele A of TO-0145125 and allele T of TO-0196109.

In all the previous methods and processes, the preferred combinations of QTLs and Tm-1 gene to be associated or detected are as disclosed in connection with the first aspect of the invention, and include namely Tm-1 homozygously with QTL2 heterozygously and QTL3 homozygously or heterozygously, as well as Tm-1 heterozygously with QTL2 heterozygously and QTL3 homozygously or heterozygously.

In view of the ability of the resistant plants of the invention to restrict the damages caused by TBRFV infection, to reduce the virus titer and to delay, reduce and/or inhibit the viral replication, and thus its propagation, they are advantageously grown in an environment infested or likely to be infested or infected by TBRFV, especially the Israeli strain or isolate; in these conditions, the resistant plants of the invention produce more marketable tomatoes than susceptible plants. They moreover restrict the spread of the virus to other fields, thus protect less resistant plants, therefore indirectly improving also their yield.

The invention is thus also directed to a method for improving the yield of tomato plants in an environment infested, or likely to be infected by TBRFV, especially the Israeli strain or isolate, comprising growing TBRFV-resistant tomato plants according to the invention, thus comprising in their genome at least one tolerance QTL, i.e. QTL1, QTL2, and/or QTL3 as defined in WO2018/219941 on chromosome 6, 9 and 11 respectively, in combination with the Tm-1 gene, either homozygously or heterozygously. Preferably at least one of the tolerance QTL is present homozygously. According to another embodiment, at least one is present heterozygously, preferably with another one present homozygously. Preferably, the method comprises a first step of choosing or selecting a tomato plant having at least one of the tolerance QTLs and the Tm-1 gene. The method can also be defined as a method of increasing the productivity of a tomato field, tunnel, greenhouse or glasshouse.

As disclosed in the preceding aspect, the tomato plant to be grown preferably also comprises a Tm-2 or Tm-2² allele, preferably heterozygously. The preferred genotypes of the tomato plant or seed to be grown are illustrated in table 1.

According to a preferred embodiment, the method comprises growing a tomato plant comprising QTL3 as defined above on chromosome 11, preferably homozygously, and a Tm-1 gene. The invention is also directed to a method for reducing the loss on tomato production in condition of TBRFV infestation or infection, comprising growing a TBRFV-resistant tomato plant as defined above.

These methods are particularly valuable for a population of tomato plants, either in a field, in tunnels, greenouses or in glasshouses.

Alternatively, said methods for improving the yield or reducing the loss on tomato production may comprise a first step of identifying tomato plants resistant to TBRFV and comprising in their genome a tolerance QTL on chromosome 6, 9 and/or 11, homozygously or heterozygously, in combination with a Tm-1 gene, and then growing said resistant plants in an environment infested or likely to be infested by the virus. Preferably, the plants comprise homozygously a tolerance QTL on chromosome 11 in combination with the Tm-1 gene, the tolerance QTL on chromosome 9 heterozygously and the Tm-2 or Tm-2² allele heterozygously.

According to a preferred embodiment, the plants to be identified at the first step comprise allele G of TO-0182276.

The resistant plants of the invention are also able to restrict and even inhibits the growth of TBRFV, especially the Israeli isolate or strain of TBRFV, thus limiting the infection of further plants and the propagation of the virus. Accordingly, the invention is also directed to a method of protecting a field, tunnel, greenhouse or glasshouse, or any other type of plantation, from TBRFV infestation, or of at least limiting the level of infestation by TBRFV of said field, tunnel, greenhouse or glasshouse or of limiting the spread of TBRFV in a field, tunnel, greenhouse or glasshouse, especially in a tomato field. Such a method preferably comprises the step of growing a resistant plant of the invention, i.e. a plant comprising in its genome a tolerance QTL on chromosome 6, 9 and/or 11, preferably homozygously, and the Tm-1 gene. The plant of the invention to be used preferably comprises QTL3 on chromosome 11; more preferably the plant exhibits allele G of TO-0182276. Other preferred resistant plants have one of the genomic combinations disclosed in table 1.

Preferably, the method comprises a first step of choosing or selecting a tomato plant having a tolerance QTL, especially QTL3 on chromosome 11, and Tm-1 resistance gene.

The methods may also comprise a subsequent step of harvesting tomatoes.

The invention also concerns the use of a plant resistant to TBRFV for controlling TBRFV infection or infestation in a field, tunnel, greenhouse or glasshouse, or other plantation; such a plant is a plant of the invention, comprising in its genome at least one of the tolerance QTL as defined above, preferably homozygously, on chromosomes 6, 9 and/or 11 and the Tm-1 gene. In a preferred embodiment, the plant comprises in its genome two tolerance QTLs, at least one heterozygously, for example one heterozygously and one homozygously.

According to this use, the plants of the invention are therefore used for protecting a field, tunnel, greenhouse or glasshouse from TBRFV infestation. The plants of the invention to be used preferably comprises QTL3 on chromosome 11; more preferably they exhibit allele G of TO-0182276. Other preferred resistant plants have one of the genomic combinations disclosed in table 1. The TBRFV is according to a preferred embodiment the Israeli strain or isolate of TBRFV.

The tolerance QTLs are preferably those present in the genome of a plant of the seed HAZTBRFVRES1 NCIMB 42758.

In all these uses, the preferred combinations of QTLs and Tm-1 gene to be associated or detected are as disclosed in connection with the first aspect of the invention, and include namely Tm-1 homozygously with QTL2 heterozygously and QTL3 homozygously or heterozygously, as well as Tm-1 heterozygously with QTL2 heterozygously and QTL3 homozygously or heterozygously.

### LEGEND OF FIGURES

FIG. 1: Results of the first ELISA tests conducted at 45 DPI (“Microlab” 1st scoring at 45 DPI) illustrating the presence or absence of TBRFV coat protein in leaves of tested plants.

This figure reports the optical density, as measured at 405 nm in the ELISA test, for 4 different plants.

FIG. 2: Results of the ELISA tests conducted at 75 DPI (“Microlab” 2^{nd} scoring at 75 DPI) illustrating the presence or absence of TBRFV coat protein in leaves of tested plants.

This figure reports the optical density, as measured at 405 nm in the ELISA test, for 4 different plants.

FIG. 3: Results of the ELISA tests conducted at around 110 DPI illustrating the presence or absence of TBRFV coat protein in leaves of tested plants.

This figure reports the optical density, as measured at 405 nm in the ELISA test, for 4 different plants.

FIG. 4: Results of the ELISA tests conducted at 70 DPI on different QTLs combinations, illustrating the presence or absence of TBRFV coat protein in leaves of tested plants.

FIG. 5: Results of the ELISA tests conducted at 91 DPI on different QTLs combinations, illustrating the presence or absence of TBRFV coat protein in leaves of tested plants.

FIG. 6: Results of the evaluation of foliar symptoms at 31 days post ToBRFV inoculation.

Ch11-S, Ch11-H, Ch11-R means respectively absence of QTL3 on chromosome 11 (S), presence heterozygously of said QTL3 (H), or presence homozygously of said QTL3 (R).

Ch9-S, Ch9-H, Ch9-R means respectively absence of QTL2 on chromosome 9 (S), presence heterozygously of said QTL2 (H), or presence homozygously of said QTL2 (R).

Tm1-S, Tm1-H, Tm1-R means respectively absence of Tm-1 gene on chromosome 2 (S), presence heterozygously of the Tm1 gene (H), or presence homozygously of said Tm1 gene (R).

FIG. 7: Results of the ELISA tests conducted at 35 DPI on different QTLs combinations, illustrating the presence or absence of TBRFV coat protein in leaves of tested plants.

This figure reports the optical density, as measured at 405 nm in the ELISA test. The QTLs and Tm1 gene combinations are as explained for FIG. 6.

FIG. 8: Results of the evaluation of fruit symptoms at 112 days post ToBRFV inoculation.

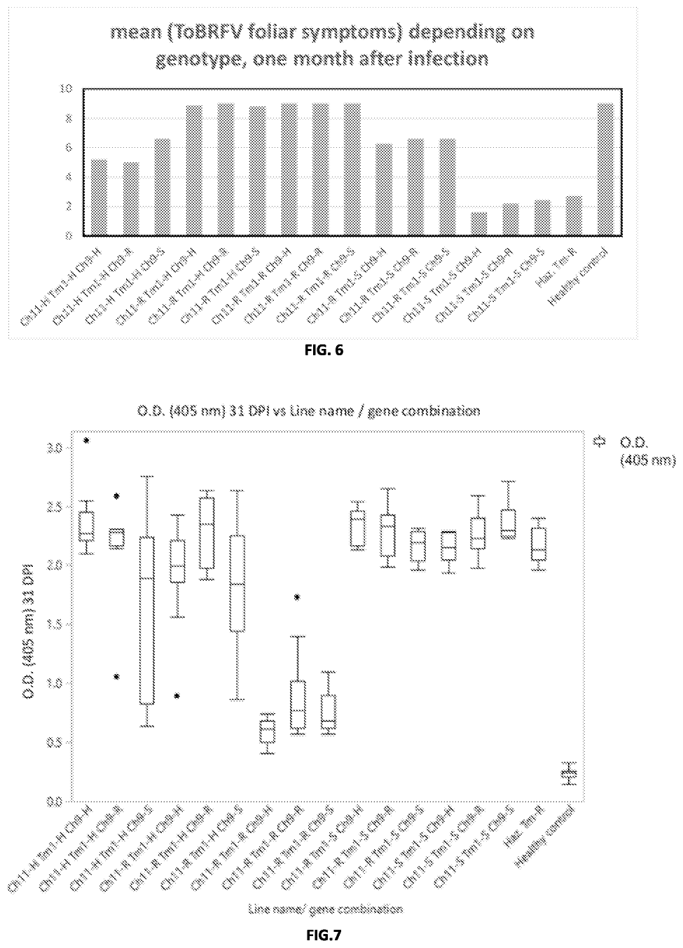

The genotypes tested are as detailed for FIG. 6.

### EXAMPLES

### Example 1: Material and Methods

Lines Description:

Line Haz-Tm1:

This line is a commercial indeterminate tomato of loose type with regular round and red fruits of about 120 g. The plant has light green foliage and is resistant to TMV race 0.

Test resistance: Line Haz-Tm1 was tested in 2 repeats of 10 plants each (total of 20 plants) for TBRFV resistance. The susceptible controls used were as follow (table 2):

TABLE 2

| Susceptible | | No. of | Foliar | Fruit |
|---|---|---|---|---|
| control name | Rep. | plants | symptoms | symptoms |
| HA-29628 | 1 | 10 | Severe | Light |
| HA-29628 | 2 | 10 | Severe | Light |
| HA-29406 | 1 | 10 | Severe | Severe |
| HA-29406 | 2 | 10 | Severe | Severe |

“Rep” is the number of the repeat
“No. of plants” is the number of plants in the repeat.

Line NB2: Used to Make the Population

This line is an indeterminate tomato of loose type with globe and intense red fruits of about 160 gr. The plant has dark green foliage and is resistant to Stemphylium, Verticillium, Nematode, Fol race 1 race 2, TMV race 2.

Symptoms:

The symptoms of TBRFV infection are as follows:

Mild foliar symptoms: usually mosaic which is not severe, without significant distortion of the leaflets shape.

Severe foliar symptoms: leaflets are distorted, in many cases there is also “shoestrings” symptoms, almost always mosaic is severe.

Mild fruit symptoms: some yellow lesions (sometimes looks like “blotchy” symptoms), but no misshapen, distorted fruits.

Severe fruit symptoms: typical misshapen fruits, sometimes also “chocolate spots”.

TBRFV symptoms Scoring: 4 scoring values, as described in WO2018/219941, with 4 corresponding to the absence of symptoms and 1 corresponding to severe symptoms.

ELISA Protocol:

Each sample containing 1-2 tomato leaves is crushed with homogenizer.

3 ml buffer SEB (Sample Extraction Buffer) were added and the sample is homogenized with bag mixer for 30 seconds.

The ToMV prime ELISA protocol of PrimeDiagnostics was then followed; this diagnostic test was chosen as it allows the detection of ToBRFV infection, although designed for ToMV infection.

Student's T-Test

The t-test is used to determine if the means of two sets of data are significantly different from each other.

In the comparison circles graph (see Figures), the position of the circles corresponds to the means of the various groups. The distance between the circles' centers represents the actual difference. The outside angle of intersection of the comparison circles is informative about whether the group means are significantly different.

Circles for means that are significantly different either do not intersect, or intersect slightly, so that the outside angle of intersection is less than 90 degrees.

Markers:

The SNP markers suitable for detection of tolerance QTLs are disclosed below.

Table 3: list of SNPs, their position and the alleles found in susceptible plants (1st nucleotide mentioned: S allele) vs. the alleles of the markers linked to the tolerance (2^{nd} nucleotide mentioned: T allele). Table 4: sequences of the SNPs.

TABLE 3

| SNP | Chromosome | Position SL2.40 | S/T allele |
|---|---|---|---|
| TO-0005197 | 6 | 33932438 | C/T |
| TO-0145581 | 6 | 33933905 | T/C |
| TO-0180955 | 9 | 4800680 | A/G |
| TO-0196724 | 9 | 5203457 | T/C |
| TO-0145125 | 9 | 40025769 | A/G |
| TO-0196109 | 9 | 59014540 | T/G |
| TO-0122252 | 11 | 8090264 | A/T |
| TO-0144317 | 11 | 8334467 | T/C |
| TO-0142270 | 11 | 8633469 | C/T |
| TO-0142294 | 11 | 8764030 | A/G |
| TO-0142303 | 11 | 8903092 | C/A |
| TO-0142306 | 11 | 9318832 | G/A |
| TO-0182276 | 11 | 9548029 | A/G |
| TO-0181040 | 11 | 9797143 | A/G |
| TO-0123057 | 11 | 9825111 | T/G |
| TO-0125528 | 11 | 9837711 | G/A |
| TO-0162432 | 11 | 10015478 | T/C |
| TO-0162427 | 11 | 10018811 | C/T |

TABLE 4
Sequences of the SNPs linked to the tolerance QTLs

| 'DFJ | | Sequence of the SNPs: the allele associated with the Tomato Brown |
|---|---|---|
| | SEQ ID | rugose Fruit virus tolerance in mentioned second in the bracket |
| TO-0005197 | 1 | GTCGGACCAAGAAACCATATTTGGTAACGGGTTCGAGTTGCTGCCTGAACCTTTTAGCCC[C/T] |
| | | TTGCAATATTTGTGAAGTGATATTCCTTTGTGTTATTATAATTTTTCGTTTTGAGTTTT |
| TO-0145581 | 2 | TTCAGAGAGCAACACTCCTGCAAGACCAACTCGGAGTAATTCAGTAACTCGACCTTCCAT[T/C] |
| | | TCTAGCTCTCAGTATAGTACTTACTCAAATAAATCAGGCTCTATTCTAAACACAAGCTCT |
| TO-0180955 | 3 | TTCCGAAATGAGGACGATCCATCAGCTTCTTCAGCTGAGAGCCCCTGGTC[A/G]ACATACCAG |
| | | AATTCTGTTTTTCTAAAACTGTCCAAAATCTCCTGTAAAGA |
| TO-0196724 | 4 | GATTTGAATGCCTTGCCACAGCCAGAGGATGACGA[T/C]GAGATTTTTGGACAACAATTAGAA |
| | | GATGAACCACA |
| TO-0145125 | 5 | AGAGAATGATATCACTGCCTTAGTTTCTCAATTAAAAGTTGTGCAAAAACAAAACACACA[A/G] |
| | | CTAGATGAAGAAAACAGAGCATTCGCCTCAAAGCTTCAGACAAAAGAAGTTGAGAACAAC |
| TO-0196109 | 6 | TACAATACCTTCTGGCATCCCTTTCCGCAAAACGA[T/G]AGATCTTTAGTATCAAAACCGAGAG |
| | | CACTGTCACC |
| TO-0122252 | 7 | ATGGCAATAGTGAACTGCAGATACAACTGAAATTGCAGAACACCCTTAAA[A/T]ATAGAATCA |
| | | ATAGAAAGTTGCAACAATATTTGAATGATGAAGCAACAAAG |
| TO-0144317 | 8 | AGCCATTGTGATTGTGTCTGTTGTACATTACCAAAATTCTCTAGAGAAAG[T/C]GATACACAT |
| | | GCCAGCCCTATCGATATAAAGCAACGCAAGGTGGATTCTGC |
| TO-0142270 | 9 | AACACCAGGTAGAGAGCACAGCGAAACAATGGCCTCAGGAAGATCTACTT[C/T]GCGAAGTGC |
| | | AGCAAGCCACTCCATACCTCCACCAGGCTTTGATTTCAGTG |
| TO-0142294 | 10 | TCAACTGCAACTTTAACAGCTGATTCAACTTCTTCTTCTTTCGAAACATC[A/G]CATTGAATG |
| | | TAACGACCTCCAATAGATTCAGCTAAACTTGTACCTACTTC |
| TO-0142303 | 11 | GAGGAGCTATCAACTTCATAGTCAGATTCAGAAAATGATTCAGATGAGGA[C/A]GTGGCTGAT |
| | | TCTTCTTGTTTTCTTTTCTTCCTTCTGCTCGAACTCTCTCC |
| TO-0142306 | 12 | CAGAAATAATAGAAAATCAGAAAGAAAAATCAGCTTTCTAAATGGAAAAG[G/A]CGATGGCAC |
| | | TATGTTTGAAGTTTTAAGCAACTTTTCTGAAGTCCCAAAAG |
| TO-0182276 | 13 | CTCCTATTGAACATCCTGAAAACTTGTGTCTACATCATGAGAAGATGCAGGCCAATC[A/G]CT |
| | | CAGTACATGGAATGCACGAGCATGTTAGGGGAATTCTAACGCAAAGCATAAGCTTGATACTTGA |
| | | ATAAAAGATGAAACATACTTACTTCTTCTCAAACT |
| TO-0181040 | 14 | CTCTTGGTGACAAACCACTGGCTCAATTTCTTCGCGAAGCTAAAGCTATC[A/G]CTGATGAGC |
| | | TTGTCACGGCAGGCACACGTGTCTCCTGATGAATTCAATGC |
| TO-0123057 | 15 | CATTACTGTTGAGATATCTCATCGGCAACCCCTGGAGCTTGCCCACCCGC[T/G]TGTCCTCCA |
| | | GGATCTGATTTCAGAAAGGATGAATAGTAACTGTGTTTCAG |
| TO-0125528 | 16 | CAAGAACCCAACGACTTCTTCTTCTTTGCTTATTGAAAAACTTGGTTTTGAAATGAAAGG[G/A] |
| | | ATCGAGAAATTGGATACTCAGTGGTTCTCTACTACTAAACCTTCTCCTGATTTTAAGAAA |
| TO-0162432 | 17 | TGATCGACAATTCTTGTTGTTGTTGAAACTCTGCAAGTGAGAGAGGGATG[T/C]ATATAG AG |
| | | AAAGGATATTGGTAAAGGACAATTCTAGAAGGGTCTAGGGAA |
| TO-0162427 | 18 | GCACCAGTTATAGTAATGTCCTGCTTCTTTCCTGTACCCTTATCAGTAGC[C/T]GTGACAGAA |
| | | AGAATACCGTTGGTGTCAATGTCGAACTTCACTTCAATCTG |

For Tm-1, a marker was developed based on information of Ishibashi et al, 2007: Four in-gene SNPs were defined, KASPar assays were developed and only one was found to be suitable.

Marker code: TO-0200838

Sequence of the SNP: the allele associated with the virus resistance is mentioned first (i.e. A) in the bracket:

(SEQ ID No: 21)
CAAAGCTCTT/GGAAACTTTCCTAAGTAT/AAGCTAATG[A/G]TGAAC
AGAATCTTGCTGGAGTA/GATTGGCCTTGGGGGTAGTGGAGGAACA.

KASPar Marker Primers:

Primer Forward Fam:

(SEQ ID No: 22)
GAAGGTGACCAAGTTCATGCTCAATYACTCCAGCAAGATTCTGTTCAT

Primer Forward Vic:

(SEQ ID No: 23)
GAAGGTCGGAGTCAACGGATTACTCCAGCAAGATTCTGTTCAC

Primer Reverse Common:

((SEQ ID No: 24)
CAAAGCTCTKGAAACTTTCCTAAGTA

### Example 2: Resistance Sources

First Resistance Source

The inventors have first identified a cultivated tomato (Solanum lycopersicum) line—line Haz-Tm1 as having high level of foliar resistance to TBRFV. This line was also known to contain the gene Tm-1.

According to the literature and known to the skilled breeder, the Tm-1 was initially introgressed from a wild tomato species Solanum habrochaites PI126445 into the cultivated tomato species Solanum lycopersicum view a view to imparting ToMV/TMV resistance. Resistance by this gene to ToMV was however broken within a year of its introduction to commercial tomato cultivars in 1960s. Therefore, this gene is rarely, if any, found in the currently commercial varieties and can no longer be considered as a resistance gene to ToMV or TMV.

A marker for Tm-1 gene (on chromosome 2) was developed based on the public gene sequence. Four SNPs were defined, KASPar assays were developed and only one was found to be suitable. The inventors first found that line Haz-Tm1 was highly resistant to TBRFV, in two trials under artificial laboratory test.

The inventors then later also screened the line Haz-Tm1 for fruit resistance under field conditions in greenhouse trial (natural infection). The trial was transplanted in a 4 dunam (corresponding to 4,000 m²) greenhouse. The results showed that line Haz-Tm1 exhibited mild symptoms of TBRFV on the fruits, mostly at the latest stages of the plant growth. It was concluded that line Haz-Tm1 probably has high resistance to foliar symptoms and mild and insufficient resistance to fruit symptoms.

Line Haz-Tm1 was then re-tested in tests, including ELISA test, which included:
(1) sowing in “54” trays,
(2) mechanical inoculation of young seedlings
(3) scoring—observation of Tobamoviruses symptoms
(4) Checking presence/absence of the virus with an Immunostrip kit (AGDIA) and with an ELISA test in three-point times
(5) plantlets planted in the greenhouse to full growing cycle.

Sowing in nursery trays on 9^{th} October

Mechanical inoculation: on 31^{st} October

Transplanting part of the trial in Brurim (greenhouses GH 3 and 4): 5^{th} November

Transplanting part of the trial in Mivtahinn greenhouse: 13^{th} November

1^{st} Scoring and sampling for ELISA test: 16^{th} and 17^{th} December

2^{nd} Scoring and sampling for ELISA test: 14^{th} January

3^{rd} Scoring and sampling for ELISA test: 19^{th} February.

The results of the 1^{st} scoring, around 45 days post inoculation (DPI) are detailed in table 5. At this stage, there are no fruits, thus only the foliar resistance is assayed.

TABLE 5
1^{st} scoring of foliar symptoms at 45 DPI

| | | | Total | | mild | Severe | |
|---|---|---|---|---|---|---|---|
| | | | no. of | Healthy | symptoms | symptoms | |
| No. | Line name | Location | plants | (score 4) | (score 2 or 3) | (score 1) | Remarks |
| 1 | Haz. Tm-S | GH. 3 | 5 | | | 5 | Typical |
| | | GH. 4 | 5 | | | 5 | “shoestrings” |
| | | Mivtahim | 10 | | | 10 | |
| 4 | Haz. Tm-1 | GH. 3 | 5 | 5 | | | |
| | | GH. 4 | 5 | 5 | | | |
| | | Mivtahim | 10 | 9 | | 1 | |
| 5 | Haz. Tm-22 | GH. 3 | 5 | | | 5 | Typical severe |
| | | GH. 4 | 5 | | | 5 | mosaic |
| | | Mivtahim | 10 | | | 10 | |

The results of the ELISA test are illustrated in FIG. 1.

2^{nd} Scoring

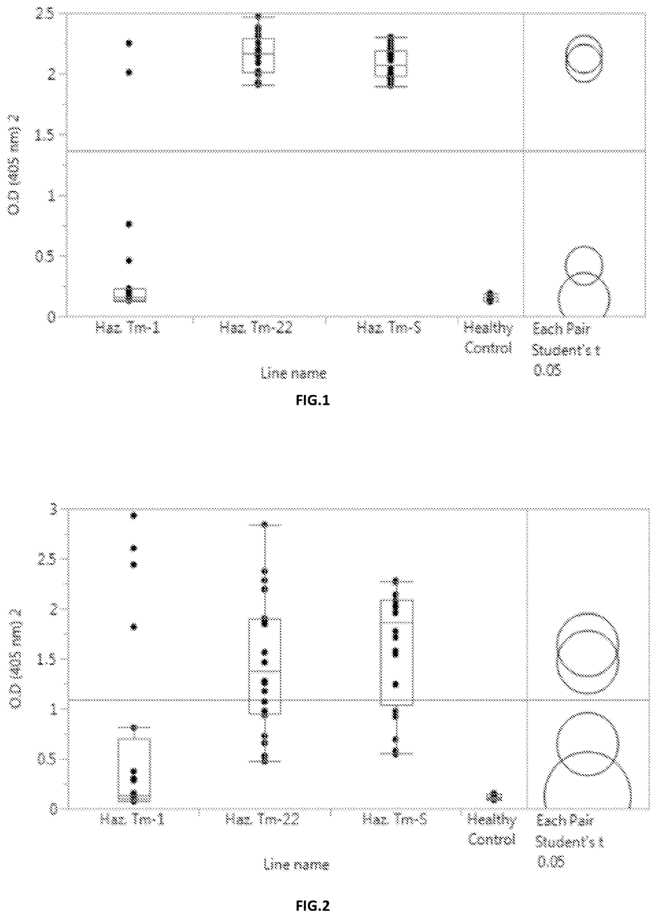

The phenotypic scoring of the 2^{nd} scoring gave similar results as obtained in the 1st scoring. The results of the ELISA test are illustrated in FIG. 2.

3^{rd} scoring

The results of the 3^{rd} scoring, around 110 DPI are detailed in table 6. At this stage, there are fruits, thus foliar and fruit resistance are scored.

TABLE 6
3^{rd} scoring of foliar symptoms at 110 DPI

| | | | | Foliar symptoms | | | Fruit symptoms | | |
|---|---|---|---|---|---|---|---|---|---|
| | | | | | Mild | | | Mild | |
| | | | Total | No | symp. | Severe | No | symp. | Severe |
| | | | no. of | symp. | (score 2 | symp. | symp. | (score 2 | symp. |
| No. | Line name | Location | plants | (score 4) | or 3) | (score 1) | (score 4) | or 3) | (score 1) |
| 1 | Haz. Tm-S | GH. 3 | 5 | | | 5 | | 5 | |
| | | GH. 4 | 5 | | | 5 | | 5 | |
| | | Mivtahim | 10 | | | 10 | | 10 | |
| 4 | Haz. Tm-1 | GH. 3 | 5 | 5 | | | 5 | | |
| | | GH. 4 | 5 | 5 | | | 5 | | |
| | | Mivtahim | 10 | | | 10 | 10 | | |
| 5 | Haz. Tm-22 | GH. 3 | 5 | | | 5 | | | 5 |
| | | GH. 4 | 5 | | | 5 | | | 5 |
| | | Mivtahim | 10 | | | 10 | | | 10 |

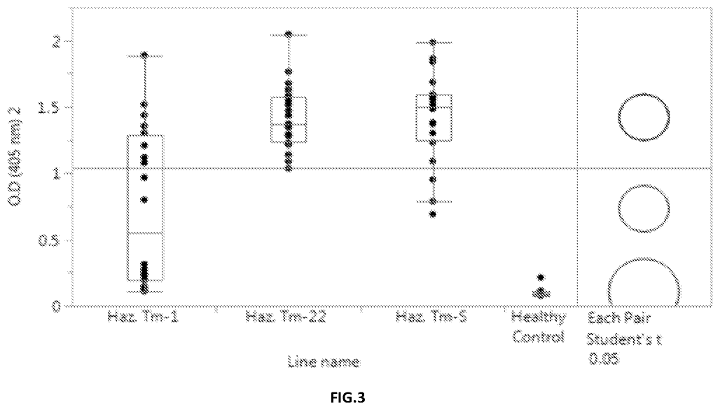

The results of the ELISA test are illustrated in FIG. 3.

The ELISA results suggest that line Haz. Tm-1 has a defense mechanism that delays the virus reproduction in the plant.

Second Resistance Source

WO2018219941 discloses tolerance QTL to TBRFV, essentially a foliar tolerance QTL, QTL3, on chromosome 11 and two fruit tolerance QTLs, QTL1 and 2, on chromosomes 6 and 9 respectively.

### Example 3: Combining by Crossing the Two Sources

Population Creation:

A cross between line Haz-Tm1 and line-NB2 was done to produce F1 seeds, the F1 was later self pollinated to produce F2 seeds. F2 seeds were sown in trays and selection for homozygous to tolerance QTL3 (i.e. QTL on chromosome 11) was done using one representative marker such as TO-0142306; these plants were advanced to produce F3 seeds, which are referred in the examples as population 1 (see table 7).

Plant Genotyping and Selection:

F3 seeds (population 1) were sown in trays, around 500 plantlets were obtained. From each F3 plantlet a leaf disc was sampled for DNA extraction and DNA was used for molecular marker analysis. For selection, two molecular markers were used, one for the TM-1 gene on chromosome 2 and the second representative of the QTL on chromosome 9 (QTL2), QTL for chromosome 11 (tolerance QTL3) was already fixed in the F2 as homozygote resistant (see population creation).

Results:

Crosses were made between line Haz. Tm-1 and one breeding line NB2 that contains the QTLs on chromosome 11 and QTL on chromosome 9. F3 seeds were obtained as disclosed above.

F3 plants were preselected in the tray using molecular markers linked to the tolerance QTLs and Tm-gene and the selected plants were mechanically inoculated at young seedlings level, plantlets were planted in the greenhouse in Bsor and grown in greenhouse.

Molecular marker analysis included one marker per QTL.

Tables 7 and 9 present different F3 plants from the population 1 containing different genotypes at the 3 loci (QTL2, QTL3 and Tm-1), the resistance based on phenotypic scoring and ELISA results of each plant. Controls are also indicated. The healthy controls were not infected.

Table 7 presents the results at 70 DPI and table 9 at 91 DPI.

Some of the foliar symptoms reported in the tables might have been increased due to the presence of pepinovirus in the greenhouse as well as severe temperature conditions. It is indeed well known that symptoms of tobamovirus infection are increased when temperature is increased. This means that the medium to severe symptoms observed in this assay, could, in milder conditions, be considered as mild symptoms only. This assay was indeed designed to be discriminative between resistant plants on one side and tolerant or susceptible plants on the other side, and not between resistant/tolerant plants and susceptible plants.

TABLE 7
ELISA results at 70 DPI, and symptoms scoring of F3 plants. R stands for Resistant
homozygote genotype, i.e. marker allele which is linked to resistance (or tolerance for the tolerance
QTLs), S stands for “susceptible homozygote genotype”. O.D.1 and O.D.2 correspond to the results
of two distinct assays. Chr11 QTL refers to tolerance QTL3; Chr9 QTL refers to tolerance QTL2.

| | | Chr11 | Tm-1 | Chr9 | O.D. | O.D. | ELISA | foliar |
|---|---|---|---|---|---|---|---|---|
| Code | Detail | QTL | Gene | QTL | (405 nm) 1 | (405 nm) 2 | result | symptoms |
| Blank | ELISA control | | | | 0.093 | 0.095 | | |
| Positive | ELISA control | | | | 1.475 | 2.196 | | |
| control | | | | | | | | |
| Negative | ELISA control | | | | 0.096 | 0.110 | | |
| control | | | | | | | | |
| Cut-off | Calculation | | | | 0.192 | 0.219 | | |
| (2*NegCntr) | | | | | | | | |
| H-1 | Healthy control | | | | 0.092 | 0.105 | negative | N.A. |
| H-2 | Healthy control | | | | 0.091 | 0.095 | negative | N.A. |
| H-3 | Healthy control | | | | 0.102 | 0.115 | negative | N.A. |
| H-4 | Healthy control | | | | 0.089 | 0.101 | negative | N.A. |
| H-5 | Healthy control | | | | 0.133 | 0.166 | negative | N.A. |
| H-6 | Healthy control | | | | 0.128 | 0.156 | negative | N.A. |
| H-7 | Healthy control | | | | 0.099 | 0.116 | negative | N.A. |
| H-8 | Healthy control | | | | 0.116 | 0.141 | negative | N.A. |
| 6305 | population 1 | R | R | R | 0.172 | 0.227 | Slightly | no |
| | | | | | | | positive | |
| 6328 | population 1 | R | R | R | 0.112 | 0.127 | negative | no |
| 6381 | population 1 | R | R | R | 0.088 | 0.100 | negative | no |
| 6415 | population 1 | R | R | R | 0.131 | 0.154 | negative | no |
| 6429 | population 1 | R | R | R | 0.093 | 0.105 | negative | no |
| 6450 | population 1 | R | R | R | 0.120 | 0.149 | negative | no |
| 6464 | population 1 | R | R | R | 0.116 | 0.143 | negative | no |
| 6470 | population 1 | R | R | R | 0.112 | 0.136 | negative | no |
| 6472 | population 1 | R | R | R | 0.111 | 0.134 | negative | no |
| 6317 | population 1 | R | R | S | 0.342 | 0.502 | slightly | no |
| | | | | | | | positive | |
| 6338 | population 1 | R | R | S | 0.311 | 0.443 | slightly | no |
| | | | | | | | positive | |
| 6339 | population 1 | R | R | S | 0.401 | 0.576 | slightly | no |
| | | | | | | | positive | |
| 6344 | population 1 | R | R | S | 0.254 | 0.352 | slightly | no |
| | | | | | | | positive | |
| 6368 | population 1 | R | R | S | 0.514 | 0.742 | slightly | no |
| | | | | | | | positive | |
| 6373 | population 1 | R | R | S | 0.289 | 0.393 | slightly | no |
| | | | | | | | positive | |
| 6386 | population 1 | R | R | S | 0.200 | 0.279 | slightly | no |
| | | | | | | | positive | |
| 6414 | population 1 | R | R | S | 0.175 | 0.262 | slightly | no |
| | | | | | | | positive | |
| 6432 | population 1 | R | R | S | 0.226 | 0.327 | slightly | no |
| | | | | | | | positive | |
| 6314 | population 1 | R | S | S | 1.365 | 2.030 | positive | mild |
| 6321 | population 1 | R | S | S | 1.307 | 1.930 | positive | mild |
| 6324 | population 1 | R | S | S | 1.444 | 2.153 | positive | mild |
| 6327 | population 1 | R | S | S | 1.689 | 2.488 | positive | mild |
| 6300 | population 1 | R | S | R | 1.462 | 2.171 | positive | Medium- |
| | | | | | | | | severe |
| 6333 | population 1 | R | S | R | 1.402 | 2.126 | positive | Medium- |
| | | | | | | | | severe |
| 6378 | population 1 | R | S | R | 1.405 | 2.091 | positive | Medium- |
| | | | | | | | | severe |
| 6380 | population 1 | R | S | R | 1.397 | 2.099 | positive | Medium- |
| | | | | | | | | severe |
| 1409-1 | population 1 | S | S | R | 1.389 | 2.086 | positive | severe |
| 1409-2 | population 1 | S | S | R | 1.434 | 2.099 | positive | severe |
| 1409-3 | population 1 | S | S | R | 1.369 | 2.084 | positive | severe |
| 1409-4 | population 1 | S | S | R | 1.642 | 2.441 | positive | severe |
| Blank | ELISA control | | | | 0.148 | 0.185 | | |
| Positive | ELISA control | | | | 1.504 | 2.228 | | |
| control | | | | | | | | |
| Negative | ELISA control | | | | 0.127 | 0.162 | | |
| control | | | | | | | | |
| Cut-off | Calculation | | | | 0.254 | 0.324 | | |
| (2*NegCnrl) | | | | | | | | |
| Haz Tm-S-1 | Susceptible | | | | 1.328 | 2.007 | positive | severe |
| | control | | | | | | | |
| Haz Tm-S-2 | Susceptible | | | | 1.421 | 2.142 | positive | severe |
| | control | | | | | | | |
| Haz Tm-S-3 | Susceptible | | | | 1.457 | 2.168 | positive | severe |
| | control | | | | | | | |
| Haz Tm-S-4 | Susceptible | | | | 1.394 | 2.094 | positive | severe |
| | control | | | | | | | |
| Haz Tm-S-5 | Susceptible | | | | 1.375 | 2.094 | positive | severe |
| | control | | | | | | | |
| Haz Tm-S-6 | Susceptible | | | | 1.386 | 2.109 | positive | severe |
| | control | | | | | | | |

TABLE 8
ELISA means of the reads at 70 DPI for the different
QTLs combination and controls:

| | No of | | | Lower | Upper |
|---|---|---|---|---|---|
| Genotype | Plants | Mean | Std Error | 95% | 95% |
| Haz Tm-S control | 6 | 2.10233 | 0.04735 | 2.0064 | 2.1983 |
| Healthy control | 8 | 0.12438 | 0.04101 | 0.0413 | 0.2075 |
| chr11-R; Tm-1-R; chr9-R | 9 | 0.14167 | 0.03866 | 0.0633 | 0.2200 |
| chr11-R; Tm-1-R; chr9-S | 9 | 0.43067 | 0.03866 | 0.3523 | 0.5090 |
| chr11-R; Tm-1-S; chr9-R | 4 | 2.12175 | 0.05799 | 2.0042 | 2.2393 |
| chr11-R; Tm-1-S; chr9-S | 4 | 2.15025 | 0.05799 | 2.0327 | 2.2678 |
| chr11-S; Tm-1-S; chr9-R | 4 | 2.17750 | 0.05799 | 2.0600 | 2.2950 |

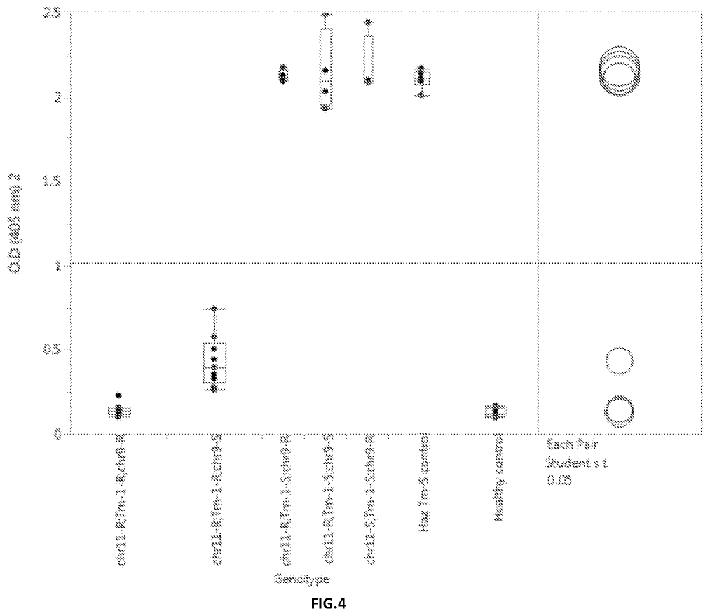

FIG. 4 illustrates the results of the ELISA test for the different QTLs combinations and controls, at 70 DPI.

It can be deduced that the combination of the Tm-1 gene and at least one of the tolerance QTL gives rise to a large decrease in the detection level of ToBRFV virus coat protein in the plants, and that the combination of the Tm-1 gene with two tolerance QTLs gives a ToBRFV detection level as low as the level found in non-infected healthy plants (Chr11-R, Tm-1-R, Chr9-R).

TABLE 9
ELISA results at 91 DPI, and symptoms scoring of F3 plants. R stands for Resistant
homozygote genotype, i.e. marker allele which is linked to resistance (or tolerance for the tolerance
QTLs), S stands for “susceptible homozygote genotype”. OD.1 and O.D.2 correspond to the results
of two distinct assays. Chr11 QTL refers to tolerant QTL3; Chr9 QTL refers to tolerant QTL2.

| | | Chr11 | Tm-1 | Chr9 | O.D. | O.D. | ELISA | foliar | Fruit | |
|---|---|---|---|---|---|---|---|---|---|---|
| Code | Detail | QTL | Gene | QTL | (405 nm) 1 | (405 nm) 2 | result | symptoms | symptoms | Remarks |
| Blank | ELISA control | | | | 0.097 | 0.110 | | | | |
| Positive | ELISA control | | | | 1.717 | 2.711 | | | | |
| control | | | | | | | | | | |
| Negative | ELISA control | | | | 0.117 | 0.137 | | | | |
| control | | | | | | | | | | |
| Cut-off | Calculation | | | | 0.232 | 0.271 | | | | |
| (2*NegCntr) | | | | | | | | | | |
| H-1 | Healthy control | | | | 0.128 | 0.165 | negative | na | na | Control for ELISA |
| H-2 | Healthy control | | | | 0.154 | 0.209 | negative | na | na | Control for ELISA |
| H-3 | Healthy control | | | | 0.111 | 0.139 | negative | na | na | Control for ELISA |
| H-4 | Healthy control | | | | 0.107 | 0.129 | negative | na | na | Control for ELISA |
| H-5 | Healthy control | | | | 0.118 | 0.146 | negative | na | na | Control for ELISA |
| H-6 | Healthy control | | | | 0.131 | 0.166 | negative | na | na | Control for ELISA |
| H-7 | Healthy control | | | | 0.119 | 0.147 | negative | na | na | Control for ELISA |
| H-8 | Healthy control | | | | 0.117 | 0.146 | negative | na | na | Control for ELISA |
| 6305 | population 1 | R | R | R | 0.385 | 0.608 | Slightly | no | no | No clear symptoms |
| | | | | | | | positive | | | but has some blotchy |
| 6328 | population 1 | R | R | R | 0.327 | 0.507 | Slightly | no | no | No clear symptoms |
| | | | | | | | positive | | | but has some blotchy |
| 6381 | population 1 | R | R | R | 0.229 | 0.342 | Slightly | no | no | No clear symptoms |
| | | | | | | | positive | | | but has some blotchy |
| 6415 | population 1 | R | R | R | 0.193 | 0.275 | Slightly | no | no | No clear symptoms |
| | | | | | | | positive | | | but has some blotchy |
| 6429 | population 1 | R | R | R | 0.264 | 0.388 | Slightly | no | no | No clear symptoms |
| | | | | | | | positive | | | but has some blotchy |
| 6450 | population 1 | R | R | R | 0.156 | 0.209 | Slightly | no | no | No clear symptoms |
| | | | | | | | positive | | | but has some blotchy |
| 6464 | population 1 | R | R | R | 0.214 | 0.308 | Slightly | no | no | No clear symptoms |
| | | | | | | | positive | | | but has some blotchy |
| 6470 | population 1 | R | R | R | 0.216 | 0.316 | Slightly | no | no | No clear symptoms |
| | | | | | | | positive | | | but has some blotchy |
| 6472 | population 1 | R | R | R | 0.407 | 0.633 | Slightly | no | no | No clear symptoms |
| | | | | | | | positive | | | but has some blotchy |
| 6317 | population 1 | R | R | S | 0.543 | 0.856 | Slightly | no | no | No clear symptoms |
| | | | | | | | positive | | | but has some blotchy |
| 6338 | population 1 | R | R | S | 0.260 | 0.400 | Slightly | no | no | No clear symptoms |
| | | | | | | | positive | | | but has some blotchy |
| 6339 | population 1 | R | R | S | 0.263 | 0.396 | Slightly | no | no | No clear symptoms |
| | | | | | | | positive | | | but has some blotchy |
| 6344 | population 1 | R | R | S | 0.206 | 0.293 | Slightly | no | no | No clear symptoms |
| | | | | | | | positive | | | but has some blotchy |
| 6368 | population 1 | R | R | S | 0.346 | 0.526 | Slightly | no | no | No clear symptoms |
| | | | | | | | positive | | | but has some blotchy |
| 6373 | population 1 | R | R | S | 0.251 | 0.377 | Slightly | no | no | No clear symptoms |
| | | | | | | | positive | | | but has some blotchy |
| 6386 | population 1 | R | R | S | 0.195 | 0.286 | Slightly | no | no | No clear symptoms |
| | | | | | | | positive | | | but has some blotchy |
| 6414 | population 1 | R | R | S | 0.280 | 0.439 | Slightly | no | no | No clear symptoms |
| | | | | | | | positive | | | but has some blotchy |
| 6432 | population 1 | R | R | S | 0.296 | 0.453 | Slightly | no | no | No clear symptoms |
| | | | | | | | positive | | | but has some blotchy |
| 6314 | population 1 | R | S | S | 0.801 | 1.259 | positive | severe | Medium | Some distortion on |
| | | | | | | | | | | fruit with pointed |
| | | | | | | | | | | blossom end |
| 6321 | population 1 | R | S | S | 0.631 | 0.982 | positive | severe | Medium | Some distortion on |
| | | | | | | | | | | fruit with pointed |
| | | | | | | | | | | blossom end |
| 6324 | population 1 | R | S | S | 0.760 | 1.203 | positive | severe | Medium | Some distortion on |
| | | | | | | | | | | fruit with pointed |
| | | | | | | | | | | blossom end |
| 6327 | population 1 | R | S | S | 0.978 | 1.558 | positive | severe | Medium | Some distortion on |
| | | | | | | | | | | fruit with pointed |
| | | | | | | | | | | blossom end |
| 6300 | population 1 | R | S | R | 0.978 | 1.593 | positive | severe | Mild | No significant |
| | | | | | | | | | | distortion, no pointed |
| | | | | | | | | | | blossom end |
| 6333 | population 1 | R | S | R | na | na | na | na | na | Dead plant |
| 6378 | population 1 | R | S | R | 0.985 | 1.594 | positive | severe | Mild | No significant |
| | | | | | | | | | | distortion, no pointed |
| | | | | | | | | | | blossom end |
| 6380 | population 1 | R | S | R | 1.083 | 1.751 | positive | severe | Mild | No significant |
| | | | | | | | | | | distortion, no pointed |
| | | | | | | | | | | blossom end |
| 1409-1 | population 1 | S | S | R | 1.012 | 1.623 | positive | severe | No | |
| 1409-2 | population 1 | S | S | R | 0.803 | 1.278 | positive | severe | No | |
| 1409-3 | population 1 | S | S | R | 0.754 | 1.180 | positive | severe | No | |
| 1409-4 | population 1 | S | S | R | na | na | na | na | na | Dead plant |
| Blank | ELISA control | | | | 0.109 | 0.128 | | | | |
| Positive | ELISA control | | | | 1.919 | 2.830 | | | | |
| control | | | | | | | | | | |
| Negative | ELISA control | | | | 0.131 | 0.161 | | | | |
| control | | | | | | | | | | |
| Cut-off | Calculation | | | | 0.263 | 0.321 | | | | |
| (2*NegCnrl) | | | | | | | | | | |
| Haz Tm-S-1 | Susceptible | | | | 1.144 | 1.698 | positive | severe | Mild-Med. | |
| | control | | | | | | | | Sym | |
| Haz Tm-S-2 | Susceptible | | | | 1.280 | 1.890 | positive | severe | Mild-Med. | |
| | control | | | | | | | | Sym | |
| Haz Tm-S-3 | Susceptible | | | | 1.148 | 1.709 | positive | severe | Mild-Med. | |
| | control | | | | | | | | Sym | |
| Haz Tm-S-4 | Susceptible | | | | 1.049 | 1.560 | positive | severe | Mild-Med. | |
| | control | | | | | | | | Sym | |
| Haz Tm-S-5 | Susceptible | | | | 0.929 | 1.399 | positive | severe | Mild-Med. | |
| | control | | | | | | | | Sym | |
| Haz Tm-S-6 | Susceptible | | | | 0.953 | 1.428 | positive | severe | Mild-Med. | |
| | control | | | | | | | | Sym | |

TABLE 10
ELISA means of the reads for the different QTLs
combination and controls (91DPI)

| | No of | | | Lower | Upper |
|---|---|---|---|---|---|
| Genotype | Plants | Mean | Std Error | 95% | 95% |
| Haz Tm-S control | 6 | 1.61400 | 0.06391 | 1.4845 | 1.7435 |
| Healthy control | 8 | 0.15588 | 0.05535 | 0.0437 | 0.2680 |
| chr11-R; Tm-1-R; chr9-R | 9 | 0.39844 | 0.05218 | 0.2927 | 0.5042 |
| chr11-R; Tm-1-R; chr9-S | 9 | 0.44733 | 0.05218 | 0.3416 | 0.5531 |
| chr11-R; Tm-1-S; chr9-R | 4 | 1.64550 | 0.07827 | 1.4869 | 1.8041 |
| chr11-R; Tm-1-S; chr9-S | 4 | 1.25050 | 0.07827 | 1.0919 | 1.4091 |
| chr11-S; Tm-1-S; chr9-R | 4 | 1.40075 | 0.07827 | 1.2422 | 1.5593 |

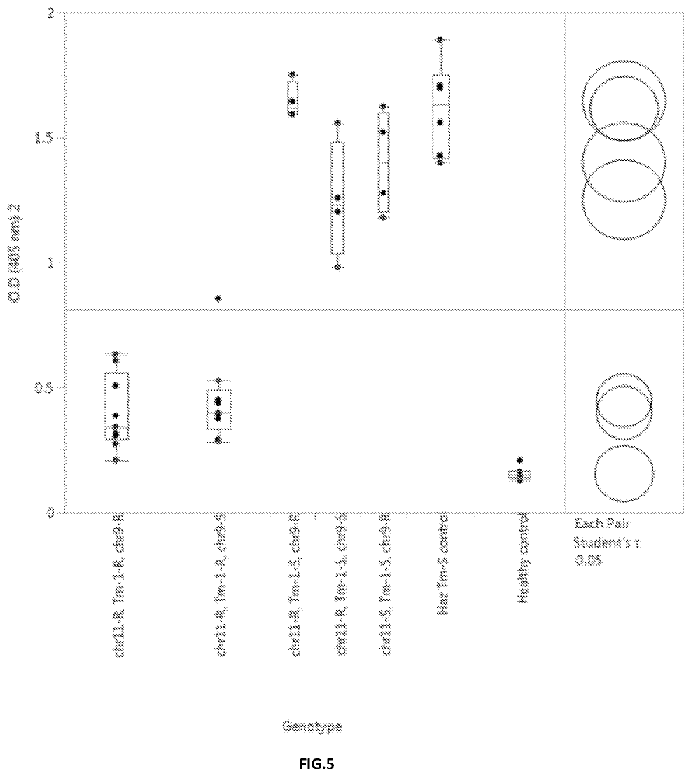

FIG. 5 illustrates the results of the ELISA test for the different QTLs combinations and controls, at 91 DPI.

Results presented in table 9 and table 10 confirm the resistance of the plants comprising Tm-1 and at least one tolerance QTL, and demonstrates that this resistance is still present 3 months after infection, thus protecting the plants from foliar and fruit damages.

### Example 4: Genetic Modification of Tomato Seeds by Ethyl Methane Sulfonate (EMS)

Seeds of a tomato varieties are to be treated with EMS by submergence of approximately 2000 seeds per variety into an aerated solution of either 0.5% (w/v) or 0.7% EMS for 24 hours at room temperature.

Approximately 1500 treated seeds per variety per EMS dose are germinated and the resulting plants are grown, preferably in a greenhouse, for example, from May to September, to produce seeds. Following maturation, M2 seeds are harvested and bulked in one pool per variety per treatment. The resulting pools of M2 seeds are used as starting material to identify the individual M2 seeds and the plants with a fruit and/or a foliar tolerance to Tomato Brown Rugose Fruit virus.

### Example 5: ToBRFV Quarantine Trail—Testing for Different Combinations of QTLs and Tm-1

In this trial, the inventors tested various combinations of the QTLs of Chromosome 11 (QTL3, or Ch11 as in example 3), of Chromosome 9 (QTL2 or Ch9 as in example 3) and Tm-1 on chromosome 2 and a susceptible control having the Tm2 gene (Haz Tm-R). The healthy control corresponds to a plant not exposed to the virus. The material and methods are as disclosed for Example 3, especially with regard to foliar and fruit notation and Elisa tests.

Time Table
Sowing: T0
Sampling for DNA extraction: T0+14 days
Mechanical inoculation: T0+28 days
Transplanting in the quarantine: T0+29 days=1 DPI
1^{st} scoring (foliar symptoms): 31 DPI
Sampling for ELISA: 35 DPI
2^{nd} scoring (fruit symptoms): 112 DPI

Results:
1^{st} scoring (foliar symptoms) & ELISA (31 DPI)

General observation: no fruit set yet, significant foliar symptoms in all the susceptible genotypes plants.

Foliar symptoms index: 1—severe foliar symptoms, 9—no visible symptoms. The mean foliar symptoms are illustrated on FIG. 6.

It is observed that the addition of the Tm-1 gene improves the scoring, and that the improvement is even better when the Tm-1 gene is present homozygously.

It is moreover observed that 3 genotypes give no symptoms, namely [Ch11-R Tm1-R Ch9-H], [Ch11-R Tm1-R Ch9-R] and [Ch11-R Tm1-R Ch9-S]. At this early stage (31 DPI) the QTL2 on chromosome 9 (fruit tolerance QTL) does not contribute to the foliar resistance.

Three other genotypes, namely [Ch11-R Tm1-H Ch9-H], [Ch11-R Tm1-H Ch9-R] and [Ch11-R Tm1-H Ch9-S] present also an important foliar resistance, although slightly less important than the 3 previous genotypes.

Elisa test was carried out four days after, at 35 DPI.

The results are reported in table 11 and illustrated in FIG. 7.

TABLE 11
Elisa results (OD at 405 nm)

| | Nb of | | | Std Err | | |
|---|---|---|---|---|---|---|
| Line/combination | plants | Mean | Std Dev | Mean | Lower 95% | Upper 95% |
| Ch11-H Tm1-H Ch9-H | 11 | 2.3586 | 0.27 | 0.0808133 | 2.1785367 | 2.5386633 |
| Ch11-H Tm1-H Ch9-R | 10 | 2.16279 | 0.407358 | 0.1288179 | 1.8713787 | 2.4541913 |
| Ch11-H Tm1-H Ch9-S | 10 | 1.71319 | 0.729089 | 0.2305581 | 1.1916314 | 2.2347486 |
| Ch11-R Tm1-H Ch9-H | 14 | 1.96077 | 0.388241 | 0.1037619 | 1.736604 | 2.1849318 |
| Ch11-R Tm1-H Ch9-R | 11 | 2.3096 | 0.277721 | 0.083736 | 2.1230245 | 2.4961755 |
| Ch11-R Tm1-H Ch9-S | 10 | 1.81148 | 0.588321 | 0.1860436 | 1.3906202 | 2.2323398 |
| Ch11-R Tm1-R Ch9-H | 10 | 0.59897 | 0.109245 | 0.0345463 | 0.5208209 | 0.6771191 |
| Ch11-R Tm1-R Ch9-R | 10 | 0.88279 | 0.38208 | 0.1208242 | 0.6094617 | 1.1561083 |
| Ch11-R Tm1-R Ch9-S | 10 | 0.75813 | 0.176183 | 0.0557138 | 0.6320965 | 0.8841635 |
| Ch11-R Tm1-S Ch9-H | 11 | 2.34288 | 0.143215 | 0.043181 | 2.2466685 | 2.4390951 |
| Ch11-R Tm1-S Ch9-R | 10 | 2.29042 | 0.222169 | 0.070256 | 2.131485 | 2.449345 |
| Ch11-R Tm1-S Ch9-S | 10 | 2.17247 | 0.134591 | 0.0425615 | 2.0761842 | 2.2687458 |
| Ch11-S Tm1-S Ch9-H | 10 | 2.14809 | 0.130071 | 0.0411319 | 2.0550431 | 2.2411369 |
| Ch11-S Tm1-S Ch9-R | 10 | 2.26306 | 0.185008 | 0.0585046 | 2.1307135 | 2.3954065 |
| Ch11-S Tm1-S Ch9-S | 7 | 2.37161 | 0.170053 | 0.064274 | 2.2143342 | 2.5288801 |
| Haz. Tm-R | 14 | 2.16388 | 0.143211 | 0.0382747 | 2.0811876 | 2.2465624 |
| Healthy control | 8 | 0.23817 | 0.049716 | 0.0175773 | 0.196605 | 0.2797325 |

It is observed that the plants having both QTL3 and Tm1 homozygously (Ch11-R Tm1-R) present a lower level of virus than all other genotypes.

From these foliar symptom scoring and ELISA tests at 31/35 DPI, it can be concluded that:
1. All plants having the Ch11-R—Tm-1-R combinations (3 combinations with Ch9 (QTL2) in all three states) are symptomless. These combinations are ELISA positive, but show a much lower virus content than all the other combinations.
2. Three combinations of Chr-11-R Tm-1-H give plants which are almost completely symptomless, but their ELISA does not appear statistically different from the susceptible genotypes at this specific stage of 30 DPI.

2^{nd} Scoring—Fruit Symptoms (112 DPI)

General observation: in most of the plants, few clusters with red fruits.

Fruit symptoms index: 1—severe foliar symptoms, 9—no visible symptoms.

Foliar symptoms are consistent with the 1^{st} observation.

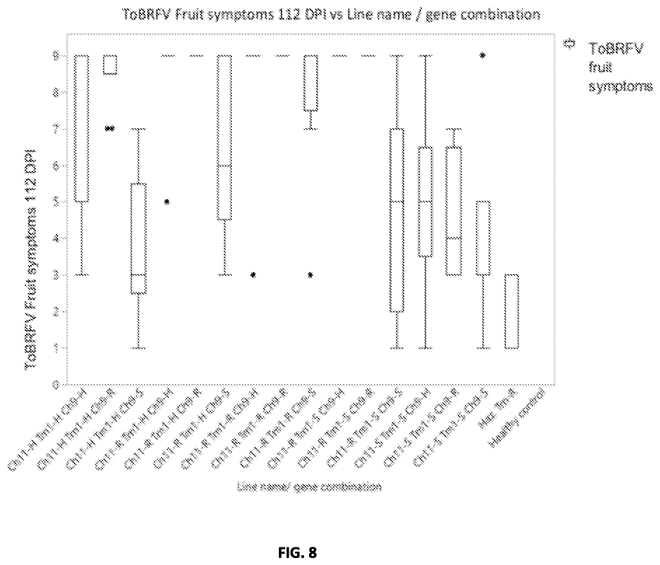

The fruit symptoms are reported on FIG. 8 and detailed in table 12.

TABLE 12
ToBRFV fruit symptoms at 112 DPI.

| | Nb of | | | Std Err | | |
|---|---|---|---|---|---|---|
| Line/combination | plants | Mean | Std Dev | Mean | Lower 95% | Upper 95% |
| Ch11-H Tm1-H Ch9-H | 11 | 7.545455 | 2.207426 | 0.6655638 | 6.062486 | 9.028423 |
| Ch11-H Tm1-H Ch9-R | 10 | 8.6 | 0.843274 | 0.2666667 | 7.996758 | 9.203242 |
| Ch11-H Tm1-H Ch9-S | 10 | 3.8 | 2.149935 | 0.6798693 | 2.262029 | 5.337971 |
| Ch11-R Tm1-H Ch9-H | 14 | 8.714286 | 1.069045 | 0.2857143 | 8.097038 | 9.331534 |
| Ch11-R Tm1-H Ch9-R | 11 | 9 | 0 | 0 | 9 | 9 |
| Ch11-R Tm1-H Ch9-S | 10 | 6.4 | 2.503331 | 0.7916228 | 4.609225 | 8.190775 |
| Ch11-R Tm1-R Ch9-H | 10 | 8.4 | 1.897367 | 0.6 | 7.042706 | 9.757294 |
| Ch11-R Tm1-R Ch9-R | 10 | 9 | 0 | 0 | 9 | 9 |
| Ch11-R Tm1-R Ch9-S | 8 | 8 | 2.13809 | 0.7559289 | 6.212512 | 9.787488 |
| Ch11-R Tm1-S Ch9-H | 11 | 9 | 0 | 0 | 9 | 9 |
| Ch11-R Tm1-S Ch9-R | 10 | 9 | 0 | 0 | 9 | 9 |
| Ch11-R Tm1-S Ch9-S | 9 | 4.555556 | 2.962732 | 0.9875772 | 2.278199 | 6.832913 |
| Ch11-S Tm1-S Ch9-H | 8 | 5 | 2.390457 | 0.8451543 | 3.001528 | 6.998472 |
| Ch11-S Tm1-S Ch9-R | 4 | 4.5 | 1.914854 | 0.9574271 | 1.45304 | 7.54696 |
| Ch11-S Tm1-S Ch9-S | 7 | 4.142857 | 2.544836 | 0.9618576 | 1.789276 | 6.496438 |
| Haz. Tm-R | 11 | 1.545455 | 0.934199 | 0.2816715 | 0.917851 | 2.173058 |

It can be deduced that the presence of the QTL2 on chromosome 9 (Ch9), either homozygously or heterozygously, greatly improves the fruit resistance (see for example the first 3 genotypes on FIG. 8, where the fruit symptoms are absent with QTL2 at the homozygous state (Ch9-R) and mild when QTL2 is present heterozygously (Ch9-H), whereas these symptoms are more important when QTL2 is absent (Ch9-S).

This assay does not allow the discrimination between the genotypes [Ch11-R, Tm1-R/H, Ch9-H] and [Ch11-R, Tm1-S, Ch9-H] as both genotypes are scored at 9 in this protocol. The results of Example 3 however suggest that, on different conditions of ToBRFV infection or at a later stage of infection, the presence of Tm1 gene, either homozygously or heterozygously, provides an enhanced level of resistance with respect to plants corresponding to the genotype [Ch11-R, Ch9-H].

### CONCLUSIONS

The presence of QTL3 on chromosome 11, preferably homozygously, in combination with Tm1 gene, provides the best foliar resistance, associated with a reduced virus titer when both are present homozygously (Ch11-R; Tm1-R).

This combination thus provides the best resistance at the first steps of ToBRFV infection; moreover, the foliar resistance ensures an adequate development of the plants, thus healthy plants with a better photosynthesis and an expected better fruit yield. In addition, the reduced virus titer implies that the plants are less likely to contaminate other surrounding plants and to propagate the virus, and the slower progression of the virus may allow to escape the more severe stages of infection, especially in case of late infection.

In order to ensure a good fruit resistance, this combination must preferably be combined with the QTL2 on chromosome 9 (Ch9-H or Ch9-R).

In conclusion, the genotypes corresponding to Ch11-R, Tm1-R and Ch9-H or R, provide globally the best results on the combined criteria of foliar resistance (advantageous for photosynthesis and yield), virus titer (less contamination and slower progression) and fruit resistance (increased yield of marketable fruits).

1. A Solanum lycopersicum plant resistant to Tomato Brown Rugose Fruit virus (TBRFV) comprising in its genome the combination of:
a. the Tm-1 resistance gene on chromosome 2, and
b. at least one quantitative trait locus (QTL) chosen from QTL3 on chromosome 11, QTL1 on chromosome 6 and QTL2 on chromosome 9, that independently confer to the plant foliar and/or fruit tolerance to TBRFV, wherein said QTLs are present in the genome of a plant of the seeds HAZTBRFVRES1 NCIMB accession number 42758.

2. A S. lycopersicum plant according to claim 1 comprising in its genome the combination of:
a. the Tm-1 resistance gene on chromosome 2, preferably homozygously,
b. said QTL3 on chromosome 11 homozygously, and
c. said QTL2 on chromosome 9 heterozygously.

3. A S. lycopersicum plant according to claim 1 comprising in its genome the combination of the Tm-1 resistance gene and at least two QTLs chosen amongst QTL1, QTL2 and QTL3, wherein at least one of said QTLs is heterozygous.

4. A S. lycopersicum plant according to claim 1 comprising homozygously in its genome the combination of:
a. the Tm-1 resistance gene on chromosome 2, and
b. said QTL3 on chromosome 11.

5. A S. lycopersicum plant according to any one of claims 1 to 4, wherein said plant delays, reduces or inhibits the replication or multiplication of the virus or reduces the virus titer in the plant.

6. A S. lycopersicum plant according to any one of claims 1 to 5, wherein said TBRFV virus is the Israeli strain of TBRFV.

7. A S. lycopersicum plant according to any one of claims 1 to 6, further comprising the Tm-2 resistance gene, preferably heterozygously.

8. A S. lycopersicum plant according to any one of claims 1 to 7, wherein said QTLs are to be found, for QTL1, on chromosome 6, within the chromosomal region delimited by TO-0005197 (SEQ ID NO:1) and TO-015581 (SEQ ID NO:2), for QTL2, on chromosome 9, within the chromosomal region delimited by TO-0180955 (SEQ ID NO:3) and TO-0196109 (SEQ ID NO:6) and for QTL3, on chromosome 11, within the chromosomal region delimited by TO-0122252 (SEQ ID NO:7) and TO-0162427 (SEQ ID NO:18).

9. The S. lycopersicum plant according to any one of claims 1 to 8, characterized by the presence in the genome of said S. lycopersicum plant of at least one of the following alleles:
allele T of TO-0005197 and/or
allele C of TO-0145581 for the presence of QTL1,
allele G of TO-0180955 and/or
allele C of TO-0196724 and/or
allele G of TO-0145125 and/or
allele G of TO-0196109 for the presence of QTL2,
allele T of TO-0122252 and/or
allele C of TO-0144317 and/or
allele T of TO-0142270 and/or
allele G of TO-0142294 and/or
allele A of TO-0142303 and/or,
allele A of TO-0142306 and/or
allele G of TO-0182276 and/or
allele G of TO-0181040 and/or
allele G of TO-0123057 and/or
allele A of TO-0125528 and/or
allele C of TO-0162432 and/or
allele T of TO-0162427 for the presence of QTL3,
in combination with allele A of SNP marker TO-0200838 (SEQ ID No: 21).

10. The plant according to any one of claims 1 to 9, wherein said plant is a progeny of an hybrid between a plant grown from the seeds of HAZTBRFVRES1 (NCIMB accession number 42758) and a S. lycopersicum plant bearing the Tm-1 gene.

11. A cell of a S. lycopersicum plant according to any one of claims 1 to 10, comprising in its genome the combination of the Tm-1 gene and at least one of said QTL1 on chromosome 6, said QTL2 on chromosome 9 and/or said QTL3 on chromosome 11, wherein said combination confers the resistance to TBRF virus.

12. Plant part of a S. lycopersicum plant according to any one of claims 1 to 10, in particular seeds, explants, reproductive material, scion, cutting, seed, fruit, root, rootstock, pollen, ovule, embryo, protoplast, leaf, anther, stem, petiole or flowers, wherein said plant part comprises cells according to claim 11.

13. Seed of a S. lycopersicum plant, which develops into a plant resistant to TBRFV according to any one of claims 1 to 10.

14. A tissue culture of cells of the plant according to any one of claims 1 to 10, wherein the cells are derived from embryos, protoplasts, meristematic cells, callus, pollen, leaves, anthers, stems, petioles, roots, root tips, seeds, flowers, cotyledons, and/or hypocotyls, and contain in their genome said QTL1 on chromosome 6, said QTL2 on chromosome 9 and/or said QTL3 on chromosome 11 independently conferring fruit or foliar tolerance to TBRF virus, in combination with the Tm-1 gene.

15. A method for detecting and/or selecting S. lycopersicum plants resistant to TBRFV, inhibiting, reducing or delaying the replication of the virus, comprising the steps of:
a. Detecting at least one of the following markers: allele T of TO-0122252, allele C of TO-0144317, allele T of TO-0142270, allele G of TO-0142294, allele A of TO-0142303, allele A of TO-0142306, allele G of TO-0182276, allele G of TO-0181040, allele G of TO-0123057, allele A of TO-0125528, allele C of TO-0162432 and allele T of TO-0162427, and
b. Detecting the homozygous presence of the Tm-1 gene, preferably detecting allele A of SNP marker TO-0200838.

16. A method for detecting and/or selecting S. lycopersicum plants resistant to TBRFV, inhibiting, reducing or delaying the replication of the virus, said method comprising:
a) Assaying tomato plants for the combination in its genome of
the Tm-1 resistance gene on chromosome 2, and
at least one genetic marker genetically linked to a QTL chosen from QTL3 on chromosome 11, QTL1 on chromosome 6 and QTL2 on chromosome 9, independently conferring to the plant foliar and/or fruit tolerance to TBRFV,
b) Selecting a plant comprising the Tm-1 gene and the genetic marker and the chosen QTL conferring foliar and/or fruit tolerance to TBRFV,
wherein the chosen QTL and the genetic marker are to be found, for QTL1, on chromosome 6, within the chromosomal region delimited by TO-0005197 (SEQ ID NO:1) and TO-015581 (SEQ ID NO:2), for QTL2, on chromosome 9, within the chromosomal region delimited by TO-0180955 (SEQ ID NO:3) and TO-0196109 (SEQ ID NO:6) and for QTL3, on chromosome 11, within the chromosomal region delimited by TO-0122252 (SEQ ID NO:7) and TO-0162427 (SEQ ID NO:18).

17. A method for conferring resistance to TBRFV to S. lycopersicum plants, comprising the steps of:
a) Crossing a plant grown from the deposited seeds NCIMB 42758, or progeny thereof, bearing QTL1, QTL2 and/or QTL3 conferring TBRFV tolerance, and a S. lycopersicum plant, preferably devoid of said QTL(s), and bearing the Tm-1 gene,
b) Selecting a plant in the progeny thus obtained, bearing one, two or three of the QTL1, QTL2 and/or QTL3 in combination with the Tm-1 gene;
c) Optionally self-pollinating one or several times the plant obtained at step b) and selecting in the progeny thus obtained a plant having resistance to TBRFV,
wherein said resistance delays, reduces or inhibits the replication or multiplication of the virus.

18. A method for conferring resistance to TBRFV to S. lycopersicum plants, comprising the steps of:
a1) Crossing a plant grown from the deposited seeds NCIMB 42758 or progeny thereof, bearing QTL1, QTL2 and/or QTL3 conferring TBRFV tolerance, and a S. lycopersicum plant, preferably devoid of said QTL(s), and bearing the Tm-1 gene, thus generating F1 hybrids,
a2) Selfing the F1 hybrids to create F2 population,
b) Selecting individuals in the progeny thus obtained having resistance to TBRFV, wherein said resistance delays, reduces or inhibits the replication of the virus.

19. The method of claim 17 or 18, wherein SNPs markers are used in steps b) and/or c) for selecting plants bearing QTL1, QTL2 and/or QTL3 conferring TBRFV tolerance and/or for selecting plants bearing the Tm-1 gene.

20. A method for breeding S. lycopersicum plants having resistance to TBRFV, comprising the steps of crossing a plant grown from the deposited seeds NCIMB 42758 or progeny thereof bearing QTL1, QTL2 and/or QTL3 conferring TBRFV tolerance, with a S. lycopersicum plant bearing the Tm-1 gene.

21. A S. lycopersicum plant obtainable by the method according to any one of claims 17 to 20.

22. A method for improving the yield of tomato plants in an environment infested by TBRFV comprising growing resistant tomato plants comprising in their genome the combination of:
a. the Tm-1 resistance gene on chromosome 2, and
b. at least one QTL, and preferably two QTLs, chosen from QTL3 on chromosome 11, QTL1 on chromosome 6 and QTL2 on chromosome 9, wherein said QTLs are present in the genome of a plant of the seeds HAZTBRFVRES1 NCIMB accession number 42758 and independently confer to tomatoes foliar and/or fruit tolerance to TBRFV.

23. The method according to claim 22, wherein said plant comprises the combination of the Tm-gene on chromosome 2 and a QTL on chromosome 11, wherein said QTL is present in the genome of a plant of the seeds HAZTBRFVRES1 NCIMB accession number 42758 and confers foliar tolerance to TBRFV.

24. A method for reducing the loss on tomato production in condition of TBRFV infestation, comprising growing resistant tomato plants comprising in their genome the combination of:
the Tm-1 resistance gene on chromosome 2, and
at least one QTL, and preferably two QTLs, chosen from QTL3 on chromosome 11, QTL1 on chromosome 6 and QTL2 on chromosome 9, wherein said QTLs are present in the genome of a plant of the seeds HAZTBRFVRES1 NCIMB accession number 42758 and independently confer to tomatoes foliar and/or fruit tolerance to TBRFV.

25. A method of protecting a field, tunnel, greenhouse or glasshouse of tomato plants from TBRFV infestation, comprising growing resistant tomato plants comprising in their genome the combination of:
the Tm-1 resistance gene on chromosome 2, and
at least one QTL, and preferably two QTLs, chosen from QTL3 on chromosome 11, QTL1 on chromosome 6 and QTL2 on chromosome 9, wherein said QTLs are present in the genome of a plant of the seeds HAZTBRFVRES1 NCIMB accession number 42758 and independently confer to tomatoes foliar and/or fruit tolerance to TBRFV.

26. Use of a tomato plant resistant to TBRFV for controlling TBRFV infestation of a field, tunnel, greenhouse or glasshouse, wherein said tomato plant comprises in its genome the combination of the Tm-1 resistance gene on chromosome 2, and at least one QTL, and preferably two QTLs, chosen from QTL3 on chromosome 11, QTL1 on chromosome 6 and QTL2 on chromosome 9, wherein said QTLs are present in the genome of a plant of the seeds HAZTBRFVRES1 NCIMB accession number 42758 and independently confer to tomatoes foliar and/or fruit tolerance to TBRFV.
